(19) United States
(12) Patent Application Publication
(10) Pub. No.: US 2025/0270269 A1
(43) Pub. Date: Aug. 28, 2025

(54) DUAL FUNCTION PROTEINS COMPRISING FGF21 MUTANT PROTEIN AND PHARMACEUTICAL COMPOSITION COMPRISING SAME

(71) Applicant: Yuhan Corporation, Seoul (KR)

(72) Inventors: Jun Hwan Kim, Seoul (KR); Seyoung Lim, Yongin-si (KR); Minji Seo, Seoul (KR); Hyun Ho Choi, Suwon-si (KR); Dohoon Kim, Yongin-si (KR); Mi Kyeong Ju, Suwon-si (KR); Ju-Young Park, Seoul (KR); Seul Gi Kim, Suwon-si (KR); Sangmyoun Lim, Seoul (KR); Jong Gyun Kim, Anyang-si (KR); Su Youn Nam, Seoul (KR)

(73) Assignee: Yuhan Corporation, Seoul (KR)

(21) Appl. No.: 18/919,743

(22) Filed: Oct. 18, 2024

Related U.S. Application Data
(63) Continuation of application No. 17/478,628, filed on Sep. 17, 2021, now abandoned.
Application No. 17/478,628 is a division of application No. 15/768,865, filed on Apr. 17, 2018, now Pat. No. 11,136,364.

(30) Foreign Application Priority Data
Oct. 28, 2015 (KR) 10-2015-0150576

(51) Int. Cl. C07K 14/50; A61K 38/18; C07K 14/575; C07K 14/605

(52) U.S. Cl. CPC C07K 14/50; A61K 38/1825; C07K 14/575; C07K 14/605; C07K 2319/30

(57) ABSTRACT

A dual function protein is disclosed. The dual function protein may be prepared by linking a biologically active protein and an FGF mutant protein to an Fc region of an immunoglobulin. The dual function protein has improved pharmacological efficacy, in vivo duration and protein stability. The dual function protein exhibits improved pharmacological efficacy, in vivo duration and protein stability. A pharmaceutical composition containing the dual function protein as an active ingredient may be effectively used as a therapeutic agent for diabetes, obesity, dyslipidemia, metabolic syndrome, non-alcoholic fatty liver diseases, non-alcoholic steatohepatitis or cardiovascular diseases.

### CROSS REFERENCE TO RELATED APPLICATIONS

This application is a Continuation of U.S. application Ser. No. 17/478,628 filed Sep. 17, 2021, which is a Divisional of U.S. application Ser. No. 15/768,865 filed Apr. 17, 2018, now U.S. Pat. No. 11,136,364 issued Oct. 5, 2021, which is a National Stage of International Application No. PCT/KR2016/012300 filed Oct. 28, 2016, claiming priority based on Korean Patent Application No. 10-2015-0150576 filed Oct. 28, 2015.

### SEQUENCE LISTING

The content of the electronically submitted sequence listing, file name: SEQUENCE_LISTING_AS_FILED.XML; size: 133.5 KB; and date of creation: Oct. 17, 2024, filed herewith, is incorporated herein by reference in its entirety.

### TECHNICAL FIELD

The present invention relates to a dual function protein including a biologically active protein and a fibroblast growth factor 21 (FGF21) mutant protein, and a pharmaceutical composition containing same.

### BACKGROUND ART

Glucagon-like peptide-1 (GLP-1) is an incretin hormone consisting of 31 amino acids, which is secreted by L cells in the intestinal tract when stimulated by food, etc. Its biological effects arise via intracellular signaling through the GLP-1 receptor, a G protein-coupled receptor which is expressed in target tissues such as β-cells in the pancreas, brain, etc. GLP-1 secreted in the blood has a very short half-life of less than 2 minutes, which is caused by a loss of activity due to the cleavage of amino acids at the N-terminus by the enzyme dipeptidyl peptidase-4 (DPP-4). Since GLP-1 stimulates the secretion of insulin in β-cells in the pancreas based on blood glucose level, it has a strong effect on lowering blood glucose without inducing hypoglycemia. Further, the administration of GLP-1 results in loss of body weight in various animal models and humans, which is known to be caused by reduced food intake due to its effect on appetite suppression. GLP-1 induces proliferation of β-cells and enhances the viability of β-cells by inhibiting cell death caused by glycolipid toxicity through GLP-1 receptor expressed in β-cells in the pancreas. Excessive secretion of glucagon increases blood glucose, which is known to be one of the causes of hyperglycemia in diabetics. In addition, it is known that GLP-1 acts on α-cells in the pancreas to inhibit fasting blood glucose elevation by inhibiting secretion of protein kinase A (PKA) protein-specific glucagon.

Exendin-4 is a clinically important GLP-1 receptor agonist. Exendin-4 is a polypeptide with 39 amino acid residues, and is normally produced in the salivary glands of the Gila Monster lizard. It is known that exendin-4 an amino acid sequence homology of 52% with GLP-1, and interacts with the GLP-1 receptor in mammals (Thorens et al. (1993) Diabetes 42:1678-1682). Exendin-4 has been shown to stimulate the secretion of insulin by insulin-producing cells in vitro, and the induction of insulin release by insulin-producing cells is stronger than GLP-1 under equimolar conditions. While exendin-4 strongly stimulates the secretion of insulin to decrease blood glucose levels in both rodents and humans with a duration of action longer than that of GLP-1, exendin-4 has exhibits antigenicity in mammals devoid of GLP-1 as it has unfamiliar epitopes in such animals.

The ability of GLP-1 and exendin-4 analogs (e.g., liraglutide and byetta) to improve glucose control in humans has been clinically confirmed. It has been reported that GLP-1 increases β-cell mass through the inhibition of apoptosis and induced proliferation. Furthermore, it has been also reported that GLP-1 acts as an intestinal hormone inhibiting gastric acid secretion and gastric emptying while enhancing satiety signals, thereby reducing appetite. Such effects of GLP-1 can explain the weight loss observed when GLP-1 analogs are administered to patients with type 2 diabetes. In addition, GLP-1 exhibits cardioprotective effects following ischemia in rodents.

Various attempts have been made to develop long-acting GLP-1 analogs. Clinically confirmed long-acting GLP-1 analogs include dulaglutide (WO 2005/000892) and albiglutide (WO 2003/059934). Dulaglutide is an Fc-fused GLP-1 analog, and albiglutide is an albumin-fused GLP-1 analog, both of which have pharmacokinetic profiles allowing for once weekly administration. Both drugs have excellent effects on lowering blood glucose and reducing body weight with once weekly administration, and also provide greatly improved convenience in terms of treatment when compared to byetta and liraglutide.

Meanwhile, fibroblast growth factor 21 (FGF21), synthesized in the liver, is a hormone known to play an important role in glucose and lipid homeostasis. FGF21 exhibits pharmacological actions in the liver, adipocytes, β cells of the pancreas, hypothalamus in the brain, and muscle tissues, where both an FGF21-specific receptor, i.e., FGF receptor, and β-klotho complex are expressed. It has been reported that in non-human primate and murine models of various diabetic and metabolic diseases, FGF21 can lower blood glucose levels in an insulin-independent manner, reduce body weight, and lower triglyceride and low-density lipoprotein (LDL) concentrations in the blood. Additionally, due to its effect of improving insulin sensitivity, FGF21 has potential for development as a novel therapeutic agent for diabetes and obesity (see WO2003/011213).

Accordingly, in order to develop a novel anti-diabetic drug based on FGF21, attempts have been made to improve its biological activity and in vivo stability by constructing FGF21 mutants based on the wild-type FGF21 sequence via substitution, insertion, and deletion of some amino acids (see WO2010/065439). However, as FGF21 has a very short half-life, it has proven problematic if used directly as a biotherapeutic agent (Kharitonenkov, A. et al. (2005) Journal of Clinical Investigation 115:1627-1635). The in vivo half-life of FGF21 is 1 to 2 hours in mice, and 2.5 to 3 hours in monkeys. Therefore, for FGF21 to be used in its current form as a therapeutic agent for diabetes, daily administration is required.

Various approaches have been reported in attempting to increase the in vivo half-life of FGF21 recombinant proteins. One such example is to link polyethylene glycol (PEG), i.e., a polymer material, to FGF21 to increase its molecular weight, thereby inhibiting renal excretion and increasing in vivo retention time (see WO2012/066075). Another approach attempts to improve the half-life by fusing it with a fatty acid, which binds to human albumin (see WO2012/010553). An additional example attempts to increase the half-life while maintaining pharmacological activity equivalent to that of wild-type FGF21 through the generation of an agonist antibody, which specifically binds to the human FGF receptor alone or as a complex with β-klotho (see WO2012/170438). In another example, the half-life was improved by preparing long-acting fusion proteins, in which an Fc region of IgG binds to an FGF21 molecule (see WO2013/188181).

Among the various technologies available to create long-acting drugs, Fc fusion technology is widely used because it has less of the disadvantages seen with other approaches, such as inducing an immune response or toxicity while increasing in vivo half-life. For the development of an Fc-fused FGF21 protein as a long-acting therapeutic drug, the following conditions should be satisfied.

First, the decrease of in vitro activity caused by fusion should be minimized. Both the N-terminus and C-terminus of FGF21 are involved in FGF21's activity. In this regard, it is known that the activities of FGF21 fusion proteins greatly vary depending on the location of the fusion. Accordingly, the activities of Fc-fused FGF21 fusion proteins, in which mutations are introduced into FGF21, may be altered depending on the presence/absence or location of the fusion. Second, a pharmacokinetic profile enabling administration at an interval of once per week in humans should be realized by the increase of in vivo half-life by the fusion. Third, considering that immunogenicity may be expected in most patients after administration of biopharmaceuticals, the immunogenicity risk due to a fusion linker or mutation should be minimized. Fourth, there should be no stability issues arising from the position of the fusion or the introduction of the mutation. Fifth, since undesired immune responses may occur depending on the isotypes of fused immunoglobulin, a solution to prevent such responses is necessary.

An attempt to develop a long-acting fusion protein by linking the Fc region of an immunoglobulin G (IgG) to an FGF21 molecule has already been reported (see WO 2013/188181). In the case of one Fc-FGF21 structure, where the Fc is fused to the N-terminus of the wild-type FGF21, while there is no distinct difference in in vitro activity as compared to that of the wild-type FGF21, the half-life is known to be very short due to in vivo degradation of the protein. To address this issue, there has been an attempt to improve the in vivo half-life by introducing several mutations at specific site locations of FGF21 to resist protein degradation. However, immunogenicity risk may increase with the introduction of multiple mutations. In contrast, in the case of an FGF21-Fc structure, where the Fc is fused to the C-terminus of the FGF21 molecule, it is known that there is a significant decrease in activity caused by fusion at this site when compared to the Fc-FGF21 structure.

Combined administration of GLP-1 and FGF21 may have a synergistic effect as compared with single administration depending on the action mechanisms and target tissues in the body, and potentially outstanding anti-diabetic efficacy and additional advantages are expected. The effects of combined administration of GLP-1 and FGF21 or a GLP-1/FGF21 dual function protein have been already investigated and reported (see WO 2010/142665 and WO 2011/020319).

Various problems must be solved in order to develop a dual function protein comprising GLP-1 and FGF21. Since wild-type GLP-1 and wild-type FGF21 have a very short in vivo half-life, they are required to be administered at least once daily, even if developed as therapeutic agents. Accordingly, long-acting technologies such as an Fc fusion are required in order to develop a long-acting dual function protein to improve convenience for patients. In a dual function drug for the two targets of GLP-1 and FGF21, the introduction of mutation(s) is essential to maintain the activity and in vivo stability of each drug, and problems associated with changes in activity, structure or stability caused by each mutation should be addressed. Medicinal effects for the two targets of GLP-1 and FGF21 should be well-balanced, and drug designs considering in vitro activities, pharmacokinetic profiles, pharmacological efficacy in animal models as well as clinical evaluation of efficacy in humans are required for this purpose. A dual function protein has a structure that cannot exist in a human body, and is structurally complex as compared with a fusion protein for a single target. In addition, since mutation or linker engineering is required to balance the two targets, the possibility of forming aggregate complexes may increase, and further protein engineering to prevent this may be required. Furthermore, potential immunogenicity may increase due to novel mutation sequences or complex structures, which should be addressed or avoided.

The present inventors have endeavored to improve the stability, pharmacokinetic profiles and pharmacological efficacy of dual function proteins including GLP-1 mutant proteins and FGF21 mutant proteins, and discovered that the stability, pharmacokinetic profiles and pharmacological efficacy of dual function proteins may be improved when a GLP-1 mutant protein is fused to an Fc region of an immunoglobulin and a novel FGF21 mutant protein is fused thereto, thereby accomplishing the present invention.

### DISCLOSURE OF INVENTION

### Technical Problem

An object of the present invention is to provide a dual function protein including a biologically active protein and an FGF21 mutant protein with improved pharmacokinetic parameters, high stability, low possibility of forming aggregation complexes, and reduced potential immunogenicity.

Another object of the present invention is to provide a pharmaceutical composition including the dual function protein for preventing or treating FGF21-associated disorders.

A further object of the present invention is to provide an isolated nucleic acid molecule encoding the dual function protein, an expression vector including the nucleic acid molecule, and a host cell including the expression vector.

### Solution to Problem

The present invention provides a dual function protein comprising an FGF21 mutant protein; a biologically active protein, or a mutant or fragment thereof; and an Fc region of an immunoglobulin, wherein the FGF21 mutant protein comprises at least one mutation selected from the group consisting of the following mutations (1) to (7):
(1) a substitution of amino acids at positions 98 to 101 from the N-terminus of a wild-type FGF21 protein with an amino acid sequence of EIRP (SEQ ID NO: 68);
(2) a substitution of amino acids at positions 170 to 174 from the N-terminus of a wild-type FGF21 protein with an amino acid sequence of TGLEAV (SEQ ID NO: 69);
(3) a substitution of amino acids at positions 170 to 174 from the N-terminus of a wild-type FGF21 protein with an amino acid sequence of TGLEAN (SEQ ID NO: 70);
(4) a substitution of an amino acid at position 170 from the N-terminus of a wild-type FGF21 protein with an amino acid N;
(5) a substitution of an amino acid at position 174 from the N-terminus of a wild-type FGF21 protein with an amino acid N;
(6) a substitution of an amino acid at position 180 from the N-terminus of a wild-type FGF21 protein with an amino acid E, along with one or more mutations (1) to (5) above; and
(7) a mutation of 1 to 10 amino acids for reducing immunogenicity of a wild-type FGF21 protein.

In addition, the present invention provides a pharmaceutical composition comprising the dual function protein for treating diabetes, obesity, dyslipidemia, metabolic syndrome, non-alcoholic fatty liver disease, non-alcoholic steatohepatitis or cardiovascular diseases.

Further, the present invention provides an isolated nucleic acid molecule encoding the dual function protein, an expression vector comprising the nucleic acid molecule, and a host cell comprising the expression vector.

### Advantageous Effects of Invention

A dual function protein of the present invention, prepared by linking a biologically active protein and an FGF mutant protein to an Fc region of an immunoglobulin, has improved pharmacological efficacy, in vivo duration and protein stability. In addition, a pharmaceutical composition including the dual function protein as an active ingredient can be used as a therapeutic agent for diabetes, obesity, dyslipidemia, metabolic syndrome, non-alcoholic fatty liver diseases, non-alcoholic steatohepatitis or cardiovascular diseases.

### BRIEF DESCRIPTION OF DRAWINGS

FIGS. 1A to 1C are graphs showing the in vitro activities of fusion proteins including FGF21 mutant proteins (hereinafter, “FGF21 mutant fusion protein”) using a HEK293 cell line in which human β-klotho is overexpressed. No FGF21 mutant fusion proteins exhibited a significant decrease in activity due to the introduction of a mutation.

FIGS. 2A and 2B are graphs showing the in vitro activities of FGF21 mutant fusion proteins with various linkers connecting the N-terminus of FGF21 to an Fc region, using a HEK293 cell line in which human β-klotho is overexpressed. No FGF21 mutant fusion protein exhibited a significant decrease in activity, although a slight difference was shown in activity depending on the linker sequence.

FIG. 3 is a graph showing the in vitro activities of RGE (Amgen), Fc-FGF21 (Lilly) and DFD1 using a HEK293 cell line in which human β-klotho is overexpressed. DFD1 and RGE (Amgen) had similar activities, while Fc-FGF21 (Lilly) had in vitro activity two times higher than the other proteins.

FIG. 4 shows the stability of DFD4 and that of DFD13 in order to confirm the effect of the EIRP (SEQ ID NO: 68) mutation of FGF21 on the stability of fusion protein. It was confirmed that DFD13 was associated with a lower rate of high molecular weight aggregates (HMW %) at the initial stage and at a time-point of more than 2 weeks later as compared with DFD4, indicating that the introduction of the EIRP (SEQ ID NO: 68) mutation improves the stability of the FGF21 mutant fusion protein, thereby reducing HMW % significantly.

FIG. 5 shows the concentration of each protein in the blood over time for 96 hours after subcutaneous administration of FGF21 mutant fusion proteins. Data are indicated as mean values and standard deviation.

FIG. 6 shows blood glucose levels in an ob/ob mouse model after single subcutaneous injection of DFD18, DFD72, DFD74 or Fc-FGF21 (Lilly). DFD18, DFD72 and DFD74 all had an effect of lowering blood glucose level continuously. Data are indicated as mean values and standard error of the mean (S.E.M.).

FIG. 7 shows graphs indicating the changes in body weights in the ob/ob mouse model from the day of administration to the 14th day after single subcutaneous injection of DFD18, DFD72, DFD74 or Fc-FGF21 (Lilly). DFD18, DFD72 and DFD74 all had an effect of reducing body weight as compared with the PBS-treated group. Data are indicated as mean values and standard error of the mean.

FIG. 8 shows graphs indicating the changes in glycated hemoglobin levels in the ob/ob mouse model at the day of administration (1^{st} day) and the 16^{th} day after single subcutaneous injection of DFD18, DFD72, DFD74 or Fc-FGF21 (Lilly). DFD18, DFD72 and DFD74 all reduced glycated hemoglobin levels at the 16^{th} day as compared with those at the day of administration. Data are indicated as mean values and standard error of the mean.

FIG. 9 shows blood glucose levels in an HFD/STZ mouse model after single subcutaneous injection of DFD72 or DFD74. Both DFD72 and DFD74 had the effect of lowering blood glucose level continuously. Data are indicated as mean values and standard error of the mean.

FIG. 10 shows the changes in animal body weights in the HFD/STZ mouse model from the day of administration to the 14^{th} day after single subcutaneous injection of DFD72 or DFD74. Both DFD72 and DFD74 had the effect of reducing body weight as compared with the PBS-treated group. Data are indicated as mean values and standard error of the mean.

FIG. 11 shows graphs indicating the changes in glycated hemoglobin levels in the HFD/STZ mouse model at the 1^{st} day and the 13^{th} day after single subcutaneous injection of DFD72 or DFD74. It was observed that both DFD72 and DFD74 resulted in greater reduction of glycated hemoglobin levels as compared with the PBS-treated group. Data are indicated as mean values and standard error of the mean.

FIG. 12 shows the changes in body weights measured in the diet-induced obesity mouse model from the day of administration to the 14^{th} day after single administration of DFD18. DFD18 had a significant effect on body weight reduction. Data are indicated as mean values and standard error of the mean.

FIG. 13 is a graph showing the in vitro GLP-1 activities of dual function proteins depending on the hinges which link the C-terminus of GLP-1 mutants and GLP-1 to the Fc region using a CHO cell line in which human GLP-1 receptor is overexpressed. Generally, the dual function protein including a GLP-1 (A2G) sequence (DFD23) exhibited 2 to 3 times lower activity than those of other dual function proteins including other GLP-1 mutant sequences. No significant difference in GLP-1 activities was shown between the dual function proteins including mutant sequences except the GLP-1 (A2G) sequence.

FIG. 14 shows graphs indicating the GLP-1 activities of DFD59, DFD69, DFD112 and DFD114 and the FGF21 activities of DFD69, DFD112 and DFD114. In vitro GLP-1 activities of three dual function proteins (DFD69, DFD112 and DFD114) and Fc-fused GLP-1 mutant including no FGF21 (DFD59) were measured using a CHO cell line in which human GLP-1 receptor is overexpressed. The three dual function proteins showed similar EC₅₀ values, and the Fc-fused GLP-1 mutant (DFD59) showed about 2 times higher activity than those of dual function proteins. In vitro activities of dual function proteins depending on FGF21 mutants were measured using a HEK293 cell line in which human β-klotho is overexpressed. It was confirmed that the in vitro activities of the FGF21 portion were similar in the three dual function proteins.

FIG. 15 shows the concentrations of proteins in the blood versus time for 240 hours after subcutaneous administration of dual function proteins. Data are indicated as mean values and standard deviation.

FIG. 16 shows the blood glucose levels in a db/db mouse model after single subcutaneous injection of DFD114 or DFD59 and single subcutaneous injection of combination of DFD59 and DFD74. The groups treated with dual function proteins showed stronger effects on lowering blood glucose levels than those treated with single function proteins. Data are indicated as mean values and standard error of the mean (S.E.M.).

FIG. 17 shows graphs indicating the changes in body weights in db/db mouse model from the day of administration to the 14^{th} day after single subcutaneous injection of DFD114 or DFD59 and single subcutaneous injection of combination of DFD59 and DFD74. The groups treated with dual function proteins showed stronger effects on reducing body weight than those treated with single function proteins. Data are indicated as mean values and standard error of the mean (S.E.M.).

FIG. 18 shows graphs indicating the changes in glycated hemoglobin levels in a db/db mouse model at the day of administration (1^{st} day) and the 16^{th} day after single subcutaneous injection of DFD114 or DFD59 and single subcutaneous injection of a combination of DFD59 and DFD74. The groups treated with dual function proteins showed stronger effects on reducing glycated hemoglobin levels than those treated with single function proteins or a combination thereof. Data are indicated as mean values and standard error of the mean.

FIG. 19 shows the blood glucose levels in an HFD/STZ mouse model after single subcutaneous injection of DFD114, DFD59, DFD74 or DFD72 and single subcutaneous injection of combination of DFD59 and DFD74. The groups treated with dual function proteins showed stronger effects on lowering blood glucose levels than those treated with single function proteins. Data are indicated as mean values and standard error of the mean (S.E.M.).

FIG. 20 shows the changes in body weights in the HFD/STZ mouse model from the day of administration to the 14^{th} day after single subcutaneous injection of DFD59, DFD72, DFD74 or DFD114 and single subcutaneous injection of combination of DFD59 and DFD74. The groups treated with dual function proteins showed stronger effects on reducing body weight than those treated with single function proteins. Data are indicated as mean values and standard error of the mean (S.E.M.).

FIG. 21 shows the changes in glycated hemoglobin levels in the HFD/STZ mouse model at the day of administration (1^{st} day) and the 16^{th} day after single subcutaneous injection of DFD59, DFD72, DFD74 or DFD114 and single subcutaneous injection of combination of DFD59 and DFD74. The groups treated with dual function proteins showed stronger effects on reducing glycated hemoglobin levels than those treated with single function proteins or a combination thereof. Data are indicated as mean values and standard error of the mean.

### BEST MODE FOR CARRYING OUT THE INVENTION

Hereinafter, the present invention will be described in more detail.

In an aspect, the present invention provides a dual function protein comprising a fibroblast growth factor 21 (FGF21) mutant protein; a biologically active protein, or a mutant or fragment thereof; and an Fc region of an immunoglobulin, wherein the FGF21 mutant protein comprises at least one mutation selected from the group consisting of the following mutations (1) to (7):
(1) a substitution of amino acids at positions 98 to 101 from the N-terminus of a wild-type FGF21 protein with an amino acid sequence of EIRP (SEQ ID NO: 68) (hereinafter, “EIRP”);
(2) a substitution of amino acids at positions 170 to 174 from the N-terminus of a wild-type FGF21 protein with an amino acid sequence of TGLEAV (SEQ ID NO: 69) (hereinafter, “TGLEAV”);
(3) a substitution of amino acids at positions 170 to 174 from the N-terminus of a wild-type FGF21 protein with an amino acid sequence of TGLEAN (SEQ ID NO: 70) (hereinafter, “TGLEAN”);
(4) a substitution of an amino acid at position 170 from the N-terminus of a wild-type FGF21 protein with an amino acid N;
(5) a substitution of an amino acid at position 174 from the N-terminus of a wild-type FGF21 protein with an amino acid N;
(6) a substitution of an amino acid at position 180 from the N-terminus of a wild-type FGF21 protein with an amino acid E, along with one or more mutations (1) to (5) above; and
(7) a mutation of 1 to 10 amino acids for reducing immunogenicity of a wild-type FGF21 protein.

The wild-type FGF21 protein, a hormone known to play an important role in glucose and lipid homeostasis, may be one derived from mammals such as humans, mice, pigs, monkeys, etc., preferably from humans. More preferably, the wild-type FGF21 protein may be the wild-type human FGF21 protein having an amino acid sequence represented by SEQ ID NO: 1.

The mutation included in the FGF21 mutant proteins may be, preferably, any one of the mutations of EIRP (SEQ ID NO: 68), TGLEAV (SEQ ID NO: 69), TGLEAN (SEQ ID NO: 70), G170N and G174N; a combination of any one of the mutations of TGLEAV (SEQ ID NO: 69), TGLEAN (SEQ ID NO: 70), G170N and G174N and the mutation of EIRP (SEQ ID NO: 68); a combination of any one of the mutations of EIRP (SEQ ID NO: 68), TGLEAV (SEQ ID NO: 69), TGLEAN (SEQ ID NO: 70), G170N and G174N and the mutation of A180E; or a combination of any one of the mutations of TGLEAV (SEQ ID NO: 69), TGLEAN (SEQ ID NO: 70), G170N and G174N, the mutation of EIRP (SEQ ID NO: 68) and the mutation of A180E. Furthermore, the FGF21 mutant proteins may have a conformation, in which 1 to 10 amino acids at the N-terminus or C-terminus is (are) deleted as compared to the wild-type FGF21 protein. More preferably, the FGF21 mutant proteins may include an amino acid sequence represented by any one of SEQ ID NO: 6 to 23. Still more preferably, the FGF21 mutant proteins may include an amino acid sequence represented by any one of SEQ ID NO: 6 to 23 and further have a conformation, in which 1 to 10 amino acids at the N-terminus or C-terminus is (are) deleted as compared to the wild-type FGF21 protein.

In the dual function protein, an amino acid residue N of FGF21 mutant protein introduced by a mutation may be glycosylated.

The biologically active protein may be one selected from the group consisting of insulin, C-peptide, leptin, glucagon, gastrin, gastric inhibitory polypeptide (GIP), amylin, calcitonin, cholecystokinin, peptide YY, neuropeptide Y, bone morphogenetic protein-6 (BMP-6), bone morphogenetic protein-9 (BMP-9), oxyntomodulin, oxytocin, glucagon-like peptide-1 (GLP-1), glucagon-like peptide-2 (GLP-2), irisin, fibronectin type III domain-containing protein 5 (FNDC5), apelin, adiponectin, C1q and tumor necrosis factor related protein (CTRP family), resistin, visfatin, omentin, retinol binding protein-4 (RBP-4), glicentin, angiopoietin, interleukin-22 (IL-22), exendin-4 and growth hormone. Preferably, the biologically active protein may be one selected from GLP-1, a mutant thereof and exendin-4.

The GLP-1 protein is an incretin hormone consisting of 31 amino acids, which is secreted by L cells in the intestinal tract stimulated by food, etc. For example, the GLP-1 protein may be represented by the amino acid sequence of SEQ ID NO: 42.

A mutant of GLP-1 may be represented, for example, by the amino acid sequence of any one of SEQ ID NO: 43 to 46.

As used herein, the term “Fc region,” “Fc fragment,” or “Fc” refers to a protein, which includes a heavy chain constant region 1 (CH1), a heavy chain constant region 2 (CH2) and a heavy chain constant region 3 (CH3) of an immunoglobulin, but does not include variable regions of the heavy and light chains and a light chain constant region 1 (CL1) of an immunoglobulin. Additionally, as used herein, the term “Fc region mutant” refers to one prepared by substituting part of amino acid(s) of an Fc region or by combining Fc regions of different types.

The Fc region of immunoglobulin may be an entire Fc region constituting an antibody, a fragment thereof, or an Fc region mutant. Additionally, the Fc region includes a molecule in the form of a monomer or multimer, and may further include a hinge region of the heavy chain constant region. The Fc region mutant may be modified to prevent cleavage at the hinge region. Furthermore, the hinge sequence of the Fc may have a substitution in some amino acid sequences to reduce antibody-dependent cell-mediated cytotoxicity (ADCC) or complement-dependent cytotoxicity (CDC). In addition, part of the amino acid sequence of the Fc hinge sequence may be substituted to inhibit the rearrangement of the Fab region. A lysine residue at the C-terminus of the Fc may be removed.

Preferably, the Fc region of immunoglobulin may be any one of IgG1, IgG2, IgG3, IgG4 and IgD Fc regions; or a hybrid Fc, which is a combination thereof. Further, the hybrid Fc may include an IgG4 region and an IgD region. Further, the hybrid Fc region may include part of the hinge sequence and CH2 of an IgD Fc, and CH2 and CH3 sequences of IgG4 Fc.

In addition, the Fc fragment of the present invention may be in the form of wild-type glycosylated chain, more glycosylated chain than the wild-type, less glycosylated chain than the wild-type, or deglycosylated chain. The increase, decrease, or removal of glycosylated chain may be performed by a conventional method known in the art, such as a chemical method, an enzymatic method, and a genetic engineering method using microorganisms.

Preferably, the immunoglobulin Fc region may be represented by an amino acid sequence selected from SEQ ID NO: 24 to 26, 47 and 48.

The dual function protein may include a biologically active protein, an Fc region of an immunoglobulin and an FGF21 mutant protein, linked in this order from the N-terminus to the C-terminus. Further, the dual function protein may include an FGF21 mutant protein, an Fc region of an immunoglobulin and a biologically active protein, linked in this order from the N-terminus to the C-terminus. Preferably, the dual function protein may include a biologically active protein, an Fc region of an immunoglobulin and an FGF21 mutant protein, linked in this order from the N-terminus to the C-terminus.

Furthermore, the dual function protein may include a GLP-1 mutant protein, an Fc region of an immunoglobulin and an FGF21 mutant protein, linked in this order from the N-terminus to the C-terminus. Further, the dual function protein may include an FGF21 mutant protein, an Fc region of an immunoglobulin and a GLP-1 mutant protein, linked in this order from the N-terminus to the C-terminus. Preferably, the dual function protein may include a GLP-1 mutant protein, an Fc region of an immunoglobulin and an FGF21 mutant protein, linked in this order from the N-terminus to the C-terminus.

Additionally, the dual function protein may further include a linker.

The dual function protein may be in the form, in which the FGF21 mutant protein is directly connected to the N-terminus or C-terminus of the immunoglobulin Fc region, or the FGF21 mutant protein is connected to the immunoglobulin Fc region via a linker.

In such case, the linker may be connected to the N-terminus, C-terminus, or a free radical of the Fc fragment, and also, may be connected to the N-terminus, C-terminus, or a free radical of the FGF21 mutant protein. When the linker is a peptide linker, the connection may occur in any region. For example, the linker may be connected to the C-terminus of the immunoglobulin Fc region and the N-terminus of the FGF21 mutant protein to form a fusion protein of the immunoglobulin Fc region and the FGF21 mutant protein.

Furthermore, the dual function protein of the present invention may be in the form, in which a biologically active protein is linked to the N-terminus of the Fc region of immunoglobulin of the fusion protein.

When the linker and Fc are separately expressed and then connected, the linker may be a crosslinking agent known in the art. Examples of the crosslinking agent may include 1,1-bis(diazoacetyl)-2-phenylethane, glutaraldehyde, imidoesters including N-hydroxysuccinimide ester such as 4-azidosalicylic acid and disuccinimidyl esters such as 3,3′-dithiobis(succinimidylpropionate), and bifunctional maleimides such as bis-N-maleimido-1,8-octane, but are not limited thereto.

Further, the linker may be a peptide. Preferably, the linker may be a peptide consisting of 10 to 30 amino acid residues.

Furthermore, alanine may additionally be attached to the end of linker. Preferably, the linker may be a peptide having an amino acid sequence represented by any one of SEQ ID NO: 2 to 5.

The dual function protein may be in a form in which a dimer or multimer of FGF21 mutant proteins, in which one or more FGF21 mutant proteins linked together, is connected to an immunoglobulin Fc region. Additionally, the dual function protein may be in a form of a dimer or multimer in which two or more immunoglobulin Fc regions are linked, wherein the immunoglobulin Fc regions have the FGF21 mutant protein connected thereto.

Additionally, the dual function protein may be a peptide which preferably has an amino acid sequence represented by any one of SEQ ID NO: 58 to 67. More preferably, the dual function protein may be a peptide which has an amino acid sequence represented by SEQ ID NO: 65, 66 or 67.

The FGF21 mutant protein may further include a mutation of 1 to 10 amino acids for reducing immunogenicity of the wild-type FGF21 protein. The immunogenicity may be predicted by a conventional method known in the art. For example, the potential immunogenicity of a protein may be screened by using, e.g., ITOPE™ and TCED™ methods.

Further, the mutation for minimizing the immunogenicity may be designed by a conventional method known in the art. For example, when immunogenicity is observed by performing an EPISCREEN™ analysis to evaluate potential immunogenicity, the amino acid sequences inducing the immunogenicity may be identified through T-cell epitope mapping, and the mutants with minimized immunogenicity may be designed via in silico prediction.

In another aspect, the present invention provides a pharmaceutical composition containing the dual function protein for treating FGF21-associated disorders.

As used herein, the term “FGF21-associated disorder” may include obesity, type I- and type II diabetes, pancreatitis, dyslipidemia, non-alcoholic fatty liver disease (NAFLD), non-alcoholic steatohepatitis (NASH), insulin resistance, hyperinsulinemia, glucose intolerance, hyperglycemia, metabolic syndrome, acute myocardial infarction, hypertension, cardiovascular diseases, atherosclerosis, peripheral arterial disease, apoplexy, heart failure, coronary artery heart disease, renal disease, diabetic complications, neuropathy, gastroparesis, disorder associated with a serious inactivation mutation in insulin receptor, and other metabolic disorders. Preferably, the FGF21-associated disorder may be diabetes, obesity, dyslipidemia, metabolic syndrome, non-alcoholic fatty liver disease, non-alcoholic steatohepatitis or cardiovascular diseases.

Further, the pharmaceutical composition may further include a pharmaceutical carrier. The pharmaceutical carrier may be any carrier as long as it is a non-toxic material suitable for delivering antibodies to patients. For example, distilled water, alcohol, fats, waxes and inactive solids may be included as a carrier. Pharmaceutically acceptable adjuvants (buffering agents, dispersants) may also be included in the pharmaceutical composition. In these formulations, the concentration of the dual function protein may vary greatly.

Specifically, the pharmaceutical composition may contain a formulation material for altering, maintaining, or conserving the pH, osmolarity, viscosity, transparency, color, isotonicity, odor, sterility, stability, dissolution or release rate, adsorption, or permeability of the composition. Examples of the suitable formulating material may include amino acids (e.g., glycine, glutamine, asparagine, arginine or lysine), anti-microorganism agents, anti-oxidants (e.g., ascorbic acid, sodium sulfite or sodium bisulfite), buffering agents (e.g., borate, bicarbonates, Tris-HCl, citrate, phosphate or other organic acids), bulking agents (e.g., mannitol or glycine), chelating agents (e.g., ethyelenediaminetetraacetic acid (EDTA)), complexing agents (e.g., caffeine, polyvinylpyrrolidione, β-cyclodextrin or hydroxypropyl-β-cyclodextrin), fillers, monosaccharides, disaccharides and other carbohydrates (e.g., glucose, mannose or dextrin), proteins (e.g., serum albumin, gelatin or immunoglobulin), coloring agents, flavoring agents, diluents, emulsifiers, hydrophilic polymers (e.g., polyvinylpyrrolidione), low molecular weight polypeptides, salt-forming counterions (e.g., sodium), preservatives (e.g., benzalkonium chloride, benzoic acid, salicylic acid, thimerosal, phenethyl alcohol, methylparaben, propylparaben, chlorhexidine, sorbic acid or hydrogen peroxide), solvents (e.g., glycerin, propylene glycol or polyethylene glycol), sugar alcohols (e.g., mannitol or sorbitol), suspending agents, surfactants or humectants (e.g., pluronics; PEG; sorbitan ester; polysorbate, e.g., polysorbate 20 or polysorbate 80; triton; tromethamine; lecithin; cholesterol or tyloxapol), stability improvers (e.g., sucrose or sorbitol), growth improvers (e.g., alkali metal halides, preferably, sodium chloride or potassium chloride; or mannitol, sorbitol), delivery vehicles, diluents, excipients and/or pharmaceutical adjuvants, but are not limited thereto.

In another aspect, the present invention provides a method for preventing or treating FGF21-associated disorders including administering the dual function protein to a subject in need of such prevention or treatment. This method includes, in particular, administering an effective amount of the dual function protein of the present invention to a mammal having a symptom of diabetes, obesity, dyslipidemia, metabolic syndrome, non-alcoholic fatty liver disease, non-alcoholic steatohepatitis or cardiovascular diseases which are FGF21-associated disorders.

The pharmaceutical composition of the present invention may be administered via any route. The composition of the present invention may be provided to an animal directly (e.g., topically, by administering into tissue areas by injection, transplantation, or by topical administration) or systemically (e.g., by oral- or parenteral administration) via any appropriate means. When the composition of the present invention is parenterally provided via intravenous-, subcutaneous-, ophthalmic-, intraperitoneal-, intramuscular-, oral-, rectal-, intraorbital-, intracerebral-, intracranial-, intraspinal-, intraventricular-, intrathecal-, intracistenal-, intracapsular-, intranasal-, or aerosol administration, the composition is preferably aqueous or may include a portion of a physiologically applicable body liquid suspension or solution. Accordingly, the carrier or vehicle may be added to the composition and be delivered to a patient since it is physiologically applicable. Therefore, a physiologically-appropriate saline solution may generally be included as a carrier like a body fluid for formulations.

Further, the administration frequency may vary depending on the pharmacokinetic parameters of the dual function protein in the formulations to be used. Typically, physicians would administer the composition until an administration dose to achieve a desired effect is reached. Accordingly, the composition may be administered as a unit dose, at least two doses with time intervals (may or may not contain the same amount of a target dual function protein) or administered by a continuous injection via a transplantation device or catheter. The precision of addition of an appropriate administration dose may be routinely performed by those skilled in the art, and corresponds to the scope of work being routinely performed by them.

Additionally, the preferable unit dose of the dual function protein in humans may be in a range from 0.01 μg/kg to 100 mg/kg of body weight, and more preferably from 1 μg/kg to 10 mg/kg of body weight. Although this is the optimal amount, the unit dose may vary depending on the disease to be treated or the presence/absence of adverse effects. Nevertheless, the optimal administration dose may be determined by performing a conventional experiment. The administration of the dual function protein may be performed by a periodic bolus injection, an external reservoir (e.g., an intravenous bag), or a continuous intravenous-, subcutaneous-, or intraperitoneal administration from the internal source (e.g., a bioerodable implant).

In addition, the dual function protein of the present invention may be administered to a subject recipient along with other biologically active molecules. The optimal combination of the dual function protein and other molecule(s), dosage forms, and optimal doses may be determined by a conventional experiment well known in the art.

In still another aspect, the present invention provides an isolated nucleic acid molecule encoding the dual function protein.

As used herein, the term “isolated nucleic acid molecule” refers to a nucleic acid molecule of the present invention, which is isolated from about at least 50% of proteins, lipids, carbohydrates, or other materials, discovered in nature when total nucleic acids are isolated from a source cell; which is operatively linked to a polynucleotide which is not linked in nature; or which is a part of a larger polynucleotide sequence and does not occur in nature. Preferably, in the isolated nucleic acid molecules of the present invention, there are not substantially present any other contaminated nucleic acids, or other contaminants which are discovered in the natural environment and inhibit uses of the nucleic acids in the production of polypeptides, or treatment, diagnosis, prevention, or research.

In such case, the isolated nucleic acid molecules encoding the dual function protein may have different sequences with each other due to codon redundancy. Furthermore, as long as the isolated nucleic acid can produce the dual function protein, the isolated nucleic acid may be appropriately modified, or a nucleotide may be added to the N-terminus or C-terminus of the isolated nucleic acid according to desired purposes.

The isolated nucleic acid may include, for example, a nucleotide sequence represented by any one of SEQ ID NO: 71 to 80.

In still another aspect, the present invention provides an expression vector comprising the isolated nucleic acid molecule.

As used herein, the term “expression vector” refers to a vector containing a nucleic acid sequence, which is suitable for the transformation of a host cell and directs or controls the expression of an inserted heterogenous nucleic acid sequence. The expression vector includes a linear nucleic acid, a plasmid, a phagemid, a cosmid, an RNA vector, a viral vector, and analogs thereof. Examples of the viral vector include a retrovirus, an adenovirus and an adeno-associated virus, but are not limited thereto.

As used herein, the term “expression of a heterogeneous nucleic acid sequence” or “expression” of a target protein refers to transcription of an inserted DNA sequence, translation of an mRNA transcript, and production of an Fc fusion protein product, an antibody or an antibody fragment.

A useful expression vector may be RcCMV (Invitrogen, Carlsbad) or a mutant thereof. The useful expression vector may include a human cytomegalovirus (CMV) promoter for promoting a continuous transcription of a target gene in a mammalian cell, and a bovine growth hormone polyadenylation signal sequence for enhancing the level of post-transcriptional RNA stability. In an exemplary embodiment of the present invention, the expression vector is pAD15, which is a modified vector of RcCMV.

In still another aspect, the present invention provides a host cell comprising the expression vector.

As used herein, the term “host cell” refers to a prokaryotic cell or eukaryotic cell into which a recombinant expression vector may be introduced. As used herein, the term “transformed” or “transfected” refers to introduction of a nucleic acid (e.g., a vector) into a cell by various technologies known in the art.

An appropriate host cell may be transformed or transfected with a DNA sequence of the present invention and may be used for the expression and/or secretion of the target protein. Examples of the appropriate host cell that may be used in the present invention include immortal hybridoma cells, NS/0 myeloma cells, 293 cells, Chinese hamster ovary (CHO) cells, HeLa cells, CAP cells (human amniotic fluid-derived cells), and COS cells.

Hereinafter, exemplary embodiments of the present invention will be described in detail with reference to the examples. However, these examples according to the present invention can be modified in many different forms and the scope of the present invention should not be construed as limited to the examples set forth herein.

### MODE FOR THE INVENTION

### Preparation Example 1. Preparation and Purification of Fusion Protein Containing FGF21 Mutant Protein

### Preparation Example 1-1. Preparation of Expression Vectors for Expression of FGF21 Mutant Proteins

In order to improve the stability, activity and pharmacokinetic profiles of the FGF21 in an Fc-FGF21 structure, mutation studies of FGF21 were performed.

Specifically, mutant proteins were designed for the LLLE (SEQ ID NO: 81) region (the amino acids at positions 98 to 101 from the N-terminus of the FGF21 protein) and GPSQG (SEQ ID NO: 82) region (the amino acids at positions 170 to 174 from the N-terminus of the FGF21 protein), and A180 site, which were expected to significantly affect protein activities based on 3-dimensional structure analysis of the FGF21 proteins.

The position, sequence information, target and expected effect of each mutation introduced into the FGF21 protein are listed in Table 1 below (in Table 1, N refers to glycosylated asparagine (N)). Further, FGF21 mutant proteins including the mutations described in Table 1 are listed in Table 2 below.

TABLE 1

| Se- | Posi- | Original | Mutated | | |
|---|---|---|---|---|---|
| quence | tion | sequence | sequence | Target | Expected effect |
| EIRP | 98-101 | LLLE | EIRP | Substitution | Improvement of |
| (SEQ ID | | (SEQ ID | (SEQ ID | with FGF19 | stability and |
| NO: 68) | | NO: 81) | NO: 68) | sequence | pharmacokinetics |
| TGLEAV | 170-174 | GPSQG | TGLEAV | Substitution | Improvement of |
| (SEQ ID | | (SEQ ID | (SEQ ID | with FGF19 | pharmacokinetics |
| NO: 69) | | NO: 82) | NO: 69) | sequence | |
| TGLEAN | 170-174 | GPSQG | TGLEAN | Substitution | Improvement of |
| (SEQ ID | | (SEQ ID | (SEQ ID | with FGF19 | pharmacokinetics |
| NO: 70) | | NO: 82) | NO: 70) | sequence, and | |
| | | | | addition of | |
| | | | | N-glycosylation | |
| G170N | 170 | G | N | Point | Improvement of |
| | | | | mutation, | pharmacokinetics |
| | | | | and | |
| | | | | addition of | |
| | | | | N-glycosylation | |
| G174N | 174 | G | N | Point mutation, | Improvement of |
| | | | | and addition | pharmacokinetics |
| | | | | of | |
| | | | | N-glycosylation | |
| A180E | 180 | A | E | Point mutation | Improvement of |
| | | | | | pharmacokinetics |

TABLE 2

| SEQ | |
|---|---|
| ID | |
| NO | Sequence of FGF21 mutant protein |
| 6 | FGF21 (EIRP (SEQ ID NO: 68)) |
| 7 | FGF21 (TGLEAV (SEQ ID NO: 69)) |
| 8 | FGF21 (TGLEAN (SEQ ID NO: 70)) |
| 9 | FGF21 (G170N) |
| 10 | FGF21 (G174N) |
| 11 | FGF21 (EIRP (SEQ ID NO: 68), |
| | TGLEAV (SEQ ID NO: 69)) |
| 12 | FGF21 (EIRP (SEQ ID NO: 68), |
| | TGLEAN (SEQ ID NO: 70)) |
| 13 | FGF21 (EIRP (SEQ ID NO: 68), G170N) |
| 14 | FGF21 (EIRP (SEQ ID NO: 68), G174N) |
| 15 | FGF21 (EIRP (SEQ ID NO: 68), A180E) |
| 16 | FGF21 (TGLEAV (SEQ ID NO: 69), A180E) |
| 17 | FGF21 (TGLEAN (SEQ ID NO: 70), A180E) |
| 18 | FGF21 (G170N, A180E) |
| 19 | FGF21 (G174N, A180E) |
| 20 | FGF21 (EIRP (SEQ ID NO: 68), |
| | TGLEAV (SEQ ID NO: 69), A180E) |
| 21 | FGF21 (EIRP (SEQ ID NO: 68), |
| | TGLEAN (SEQ ID NO: 70), A180E) |
| 22 | FGF21 (EIRP (SEQ ID NO: 68), |
| | G170N, A180E) |
| 23 | FGF21 (EIRP (SEQ ID NO: 68), |
| | G174N, A180E) |

Expression vectors were prepared to express the amino acids of the three components: fusion carrier, linker and FGF21 mutant in this order from the N-terminus to C-terminus. The material code of each FGF21 mutant fusion protein, sequence of mutation introduced into FGF21, sequence of fusion carrier and linker sequence are listed in Table 3 below (in Table 3, N refers to glycosylated asparagine (N)).

TABLE 3

| | | Sequence | | |
|---|---|---|---|---|
| SEQ | | of | | |
| ID | Material | FGF21 | Fusion | Linker |
| NO | code | mutation | carrier | sequence |
| 27 | DFD1 | EIRP | hyFc | C |
| | | (SEQ ID | (SEQ ID | (SEQ ID |
| | | NO: 68), | NO: 26) | NO: 2) |
| | | TGLEAV | | |
| | | (SEQ ID | | |
| | | NO: 69) | | |
| 28 | DFD3 | TGLEAV | hyFc | AKA |
| | | (SEQ ID | (SEQ ID | (SEQ ID |
| | | NO: 69) | NO: 26) | NO: 3) |
| 29 | DFD4 | TGLEAV | hyFc | GS3 |
| | | (SEQ ID | (SEQ ID | (SEQ ID |
| | | NO: 69) | NO: 26) | NO: 4) |
| 30 | DFD5 | TGLEAN | hyFc | GS3 |
| | | (SEQ ID | (SEQ ID | (SEQ ID |
| | | NO: 70) | NO: 26) | NO: 4) |
| 31 | DFD6 | G170N | hyFc | GS3 |
| | | | (SEQ ID | (SEQ ID |
| | | | NO: 26) | NO: 4) |
| 32 | DFD6 | G170N | hyFc | GS3 |
| | (E. coli) | | (SEQ ID | (SEQ ID |
| | | | NO: 26) | NO: 4) |
| 33 | DFD7 | G174N | hyFc | GS3 |
| | | | (SEQ ID | (SEQ ID |
| | | | NO: 26) | NO: 4) |
| 34 | DFD9 | none | hyFc | GS3 |
| | | | (SEQ ID | (SEQ ID |
| | | | NO: 26) | NO: 4) |
| 35 | DFD13 | EIRP | hyFc | GS3 |
| | | (SEQ ID | (SEQ ID | (SEQ ID |
| | | NO: 68), | NO: 26) | NO: 4) |
| | | TGLEAV | | |
| | | (SEQ ID | | |
| | | NO: 69) | | |
| 36 | DFD18 | EIRP | hyFc | GS3 |
| | | (SEQ ID | (SEQ ID | (SEQ ID |
| | | NO: 68), | NO: 26) | NO: 4) |
| | | TGLEAV | | |
| | | (SEQ ID | | |
| | | NO: 69), | | |
| | | A180E | | |
| 37 | DFD72 | EIRP | hyFc | GS3 |
| | | (SEQ ID | (SEQ ID | (SEQ ID |
| | | NO: 68), | NO: 26) | NO: 4) |
| | | TGLEAN | | |
| | | (SEQ ID | | |
| | | NO: 70), | | |
| | | A180E | | |
| 38 | DFD73 | EIRP | hyFc | GS3 |
| | | (SEQ ID | (SEQ ID | (SEQ ID |
| | | NO: 68), | NO: 26) | NO: 4) |
| | | G170N | | |
| 39 | DFD74 | EIRP | hyFc | GS3 |
| | | (SEQ ID | (SEQ ID | (SEQ ID |
| | | NO: 68), | NO: 26) | NO: 4) |
| | | G170N, | | |
| | | A180E | | |
| 40 | RGE | L98R, | IgG1Fc | GS3 |
| | (Amgen) | P171G, | mutant | (SEQ ID |
| | | A180E | | NO: 4) |
| 41 | Fc-FGF21 | X | IgG4Fc | GS3A |
| | (Lilly) | | mutant | (SEQ ID |
| | | | (SEQ ID | NO: 5) |
| | | | NO: 25) | |

In order to produce the FGF21 mutant fusion proteins, the nucleotide sequences encoding each of the FGF21 mutant proteins were synthesized by consulting with Bioneer Corporation (Korea) based on the amino acid sequence of each protein. NheI and NotI restriction enzyme sequences were added to the 5′ terminus and 3′ terminus of the nucleotide sequences encoding each of the FGF21 mutant proteins and an initiation codon for protein translation and a leader sequence (MDAMLRGLCCVLLLCGAVFVSPSHA) (SEQ ID NO: 83) capable of secreting the expressed protein to the outside of a cell were inserted next to the restriction enzyme sequence at the 5′ terminus. A termination codon was inserted next to the nucleotide sequence, which encodes each of the FGF21 mutant fusion proteins. The nucleotide sequence encoding each of the FGF21 mutant fusion proteins was cloned into a pTrans-empty expression vector by using the two restriction enzymes of NheI and NotI. The pTrans-empty expression vector, which has a simple structure including a CMV promoter, a pUC-derived replication origin, an SV40-derived replication origin and an ampicillin-resistant gene, was purchased from CEVEC Pharmaceuticals (Germany).

In the case of the fusion proteins of DFD6 (E. coli) and RGE (Amgen), the nucleotide sequence encoding each fusion protein was inserted into a pET30a expression vector for expression in E. coli.

### Preparation Example 1-2. Construction of Plasmid DNA for Expression of FGF21 Mutant Fusion Proteins

E. coli was transformed with each of the expression vectors constructed in Preparation Example 1-1 to obtain a large amount of plasmid DNA to be used for expression. E. coli cells, whose cell walls were weakened, were transformed with each expression vector through heat shock, and the transformants were plated out on LB plates to obtain colonies. The colonies thus obtained were inoculated into LB media, cultured at 37° C. for 16 hours, and each E. coli culture containing each expression vector was obtained in a volume of 100 mL. The E. coli thus obtained was centrifuged to remove the culture medium, and then P1, P2, P3 solutions (QIAGEN, Cat No.: 12963) were added to break the cell walls, thereby obtaining a DNA suspension in which proteins and DNAs were separated. Plasmid DNA was purified from the DNA suspension thus obtained by using a Qiagen DNA purification column. The eluted plasmid DNA was identified through an agarose gel electrophoresis, and concentrations and purities were measured by using a NANODROP™ device (Thermo scientific, NANODROP™ Lite). The DNA thus obtained was used for expression.

### Preparation Example 1-3. Expression of Fusion Proteins in CAP-T Cells

Human cell lines were transfected with each plasmid DNA type obtained in Preparation Example 1-2. Each plasmid DNA type was transduced into CAP-T cells (CEVEC), which had been cultured in PEM medium (Life technologies), by using PEI solution (Polyplus, Cat. No.: 101-10N). The mixed solution of DNA and the PEI solution was mixed with the cell suspension by using a FREESTYLE™ 293 expression medium (Invitrogen), cultured at 37° C. for 5 hours, and PEM medium was added. After culturing at 37° C. for 5-7 days, the culture was centrifuged to remove cells and a supernatant including FGF21 mutant fusion proteins was obtained.

### Preparation Example 1-4. Expression and Purification of FGF21 Mutant Fusion Proteins in E. coli

E. coli strain BL21 (DE3) was transformed with each plasmid DNA expressing DFD6 (E. coli) and RGE (Amgen) fusion proteins. The transformed E. coli expressing each fusion protein was inoculated into 20 mL of LB media, cultured at 37° C. for 15 hours with shaking, and then a portion of the culture media was inoculated into 100 mL of LB media, and cultured at 37° C. for 16 hours with shaking. Upon completion of culturing, the culture was centrifuged to obtain E. coli pellets, and then cells were disrupted using a high pressure cell disruptor to obtain inclusion bodies.

The obtained inclusion bodies were purified by washing and elution, followed by a protein refolding process. Specifically, the obtained inclusion bodies were washed 2-3 times with a buffer solution (pH 8.0) containing 0.5% Triton X-100, 50 mM Tris, 1 mM EDTA and 0.1 M NaCl to remove bacterial protein, and then resuspended in 8 M urea buffer containing 8 M urea, 50 mM Tris and 1 mM DTT. Since the proteins in 8 M urea buffer were completely denatured, a protein refolding process was performed as follows.

To begin, 8 M urea buffer was gradually diluted with 20 mM glycine buffer solution (pH 9.0) to remove urea, and from the concentration of 2 M, CuSO₄ was added to the concentration of 80 M to induce stable protein folding. The protein completing the refolding process was suspended in PBS buffer solution (pH 7.4), and the suspension was filtered with a 0.22 μm filter to remove impurities, and then loaded into a Protein A affinity chromatography column. The column was washed with 1×PBS buffer solution (pH 7.4) and then the proteins were eluted using 100 mM glycine buffer solution (pH 3.0) to prepare DFD6 (E. coli) fusion protein.

In the case of RGE (Amgen) fusion protein, the protein completing the refolding process was suspended in 50 mM Tris buffer solution (pH 8.0), the suspension was filtered with a 0.22 μm filter to remove impurities, and then loaded into an anion exchange resin column (POROS® HQ 50 μm, Thermo Fisher Scientific). The column was washed with 50 mM Tris buffer solution (pH 8.0), and then 50 mM Tris buffer solution (pH 8.0) was administered along the concentration gradient to elute RGE (Amgen) fusion protein. The RGE (Amgen) fusion protein obtained by the anion exchange resin was mixed with ammonium sulfate to the concentration of 1 M, and then purified using a hydrophobic interaction chromatography column (Phenyl sepharose FF, GE Healthcare). Specifically, the column was washed with 50 mM Tris buffer solution (pH 8.0) containing 1 M ammonium sulfate, 50 mM Tris buffer solution (pH 8.0) was administered along the concentration gradient, and the eluted fractions were analyzed through 10% Tris-glycine gel electrophoresis. The gel was dyed with coomassie brilliant blue R with mild shaking, and the fractions containing FGF21 mutant fusion protein with high purity were collected and then dialyzed overnight at 4° C. using a final buffer solution (1×PBS, 1 mM EDTA, pH 7.4). Upon completion of the dialysis, the obtained protein stock solution was concentrated at 3,000 rpm by using a 30,000 MW cut-off centrifugation filter at 4° C. The concentration of FGF21 mutant fusion protein was measured via BCA quantitative analysis.

### Preparation Example 1-5. Purification of FGF21 Mutant Fusion Proteins

Protein A affinity chromatography column (GE Healthcare) was equilibrated with 1×PBS buffer solution (pH 7.4). The culture supernatant including each FGF21 mutant fusion protein obtained in Preparation Example 1-3 was filtered with a 0.2 μm filter, and then loaded into a Protein A affinity chromatography column. The column was washed with 1×PBS buffer solution (pH 7.4) and then proteins were eluted using 100 mM glycine buffer solution (pH 3.0). The fusion proteins obtained by affinity chromatography were purified using an anion exchange resin column (POROS® HQ 50 μm, Thermo Fisher Scientific). The anion exchange resin column was equilibrated with 50 mM Tris buffer solution (pH 8.0), before the FGF21 mutant fusion proteins were eluted from the column. Specifically, after washing the column with 50 mM Tris buffer solution (pH 8.0), 50 mM Tris buffer solution (pH 8.0) was dispensed along the concentration gradient and the eluted fractions were analyzed. Each eluted fraction was analyzed using size exclusion chromatography (SEC-HPLC), and the fractions including FGF21 mutant fusion proteins with high purity were collected. The concentration and quantitative analysis were performed in accordance with the methods described in Preparation Example 1-4.

### Experimental Example 1. In Vitro Activities of Fusion Proteins

### Experimental Example 1-1. Effect of FGF21 Mutations on Protein Activity

The in vitro activities of fusion proteins DFD4, DFD5, DFD6, DFD6 (E. coli), DFD7, DFD9, DFD13, DFD18, DFD72, DFD73 and DFD74 prepared in Preparation Example 1 were measured.

Specifically, the in vitro FGF21 activities of the fusion proteins were evaluated using a HEK293 cell line (Yuhan Corporation, Korea) which was modified to overexpress human β-klotho, a coreceptor of FGF21. For the evaluation of activity, the concentrates containing the fusion proteins prepared in Preparation Examples 1-4 and 1-5 were subjected to a 3-fold serial dilution at a concentration of 3 μM. After having been cultured in a serum-deficient state for 5 hours, the cell line overexpressing human β-klotho was treated with the diluted fusion proteins for 20 minutes, and then was lysed by adding cytolysis buffer (Cisbio/Cat #64ERKPEG) with stirring at 60 rpm for 30 minutes at room temperature. The cell lysate solution was mixed with antibodies (Cisbio/Cat #64ERKPEG), which can detect extracellular signal-regulated kinase (ERK) and phosphorylated ERK, and the mixture was maintained at room temperature for 2 hours. Fluorescence was detected using a fluorometric detector (TECAN/GENiosPro). The activities of the fusion proteins were measured by comparing their EC₅₀ values.

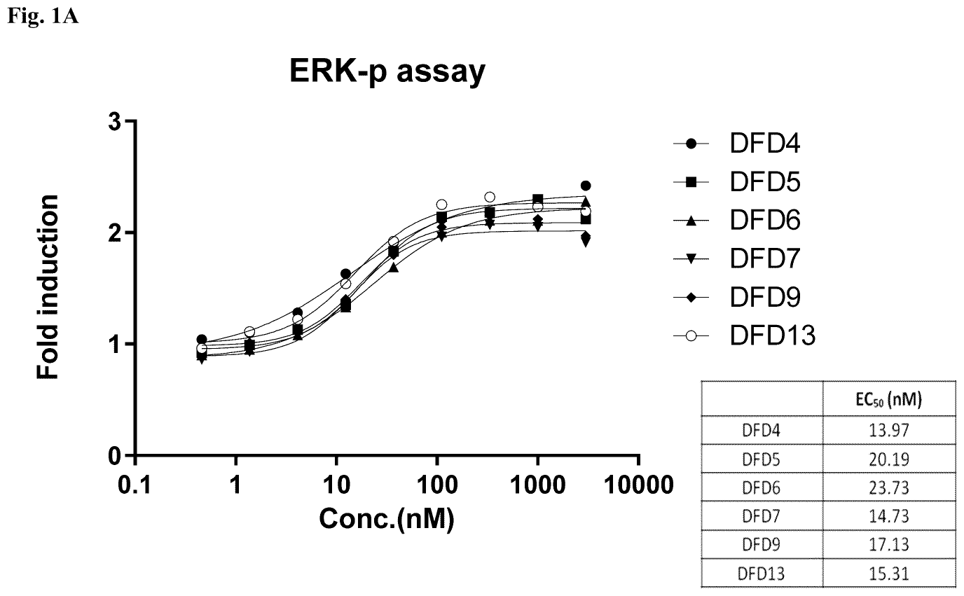

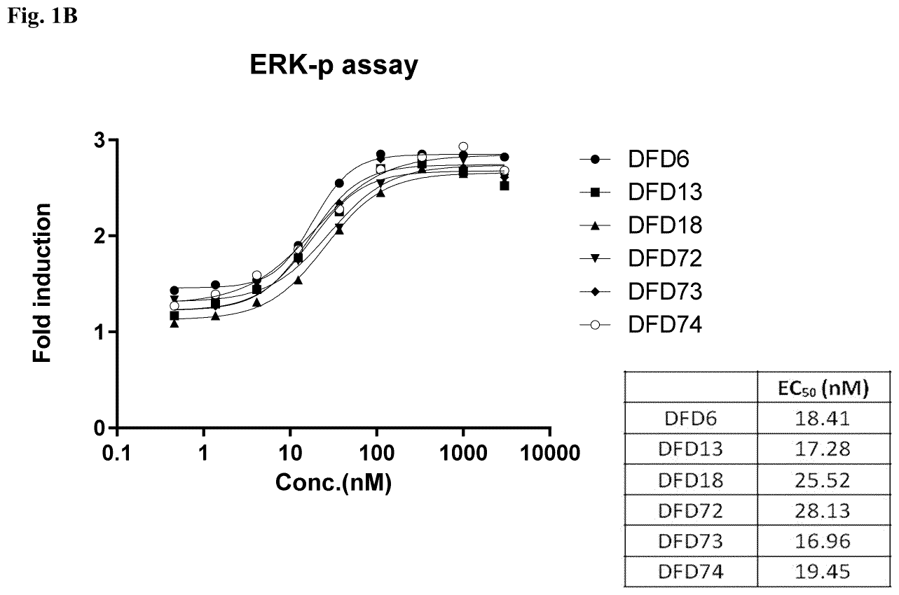

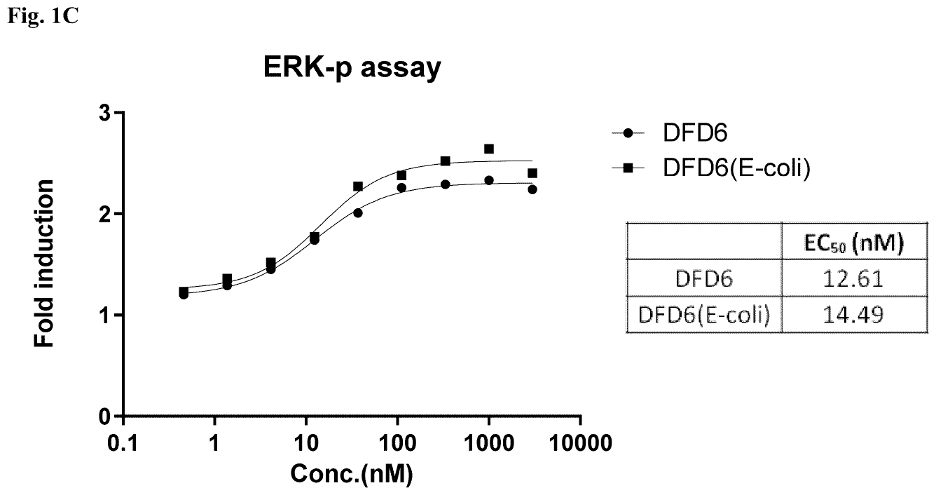

As shown in FIGS. 1A to 1C, it was confirmed that the in vitro activities of the fusion proteins prepared by introducing mutation sequences into the wild-type FGF21 protein were not inhibited, and the activities of each fusion protein were similar to each other. It was also confirmed that through the DFD6 (E. coli) sample expressed in E. coli and the DFD6 sample expressed in animal cells, the in vitro activities of the fusion proteins prepared by introducing N-glycosylation mutation into the wild-type FGF21 protein were not inhibited.

### Experimental Example 1-2. Effect of Linker Sequence on Protein Activity

The in vitro activities of fusion proteins DFD1, DFD3, DFD4 and DFD13 prepared in Preparation Example 1 were measured.

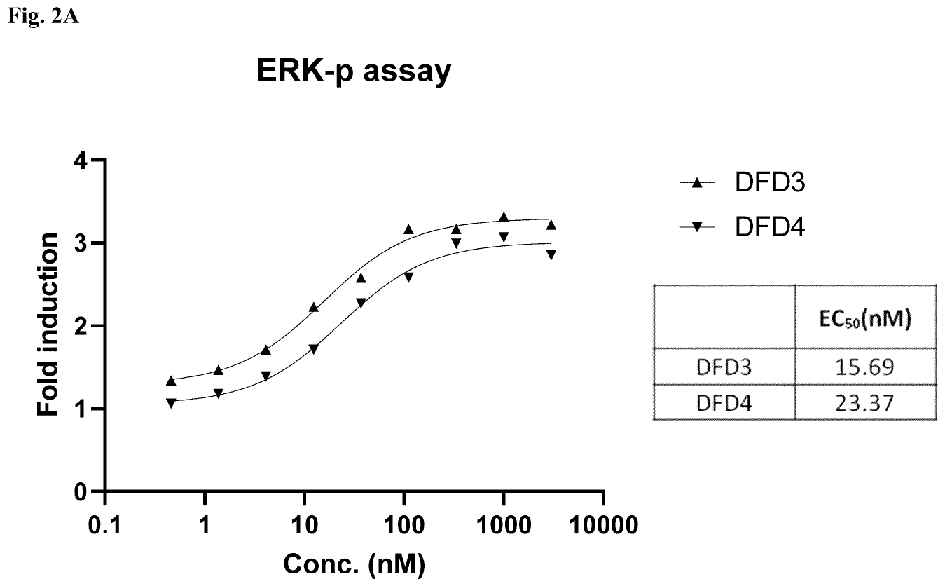

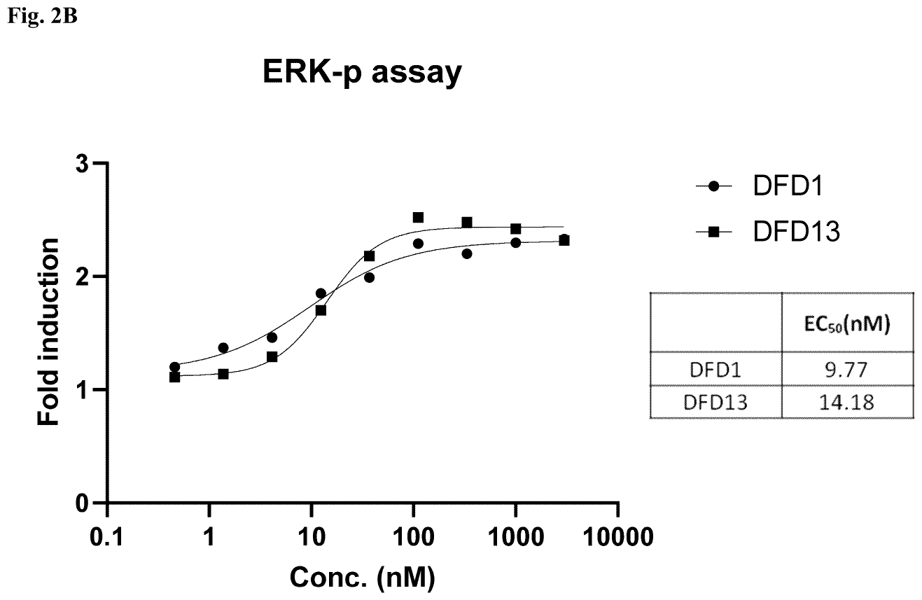

Specifically, the FGF21 activities of the fusion proteins were measured by using the concentrates containing the fusion proteins prepared in Preparation Example 1-5 in accordance with the methods described in Experimental Example 1-1. The results are shown in FIGS. 2A and 2B.

It was confirmed that no FGF21 mutant fusion protein showed a significant decrease in the activity, although a slight difference was shown in the activity depending on the linker sequence, as shown in FIGS. 2A and 2B.

### Experimental Example 1-3. Experimental Results for DFD1, RGE (Amgen) and Fc-FGF21 (Lilly)

The in vitro activities of fusion protein DFD1 prepared in Preparation Example 1 and control proteins RGE (Amgen) and Fc-FGF21 (Lilly) were measured.

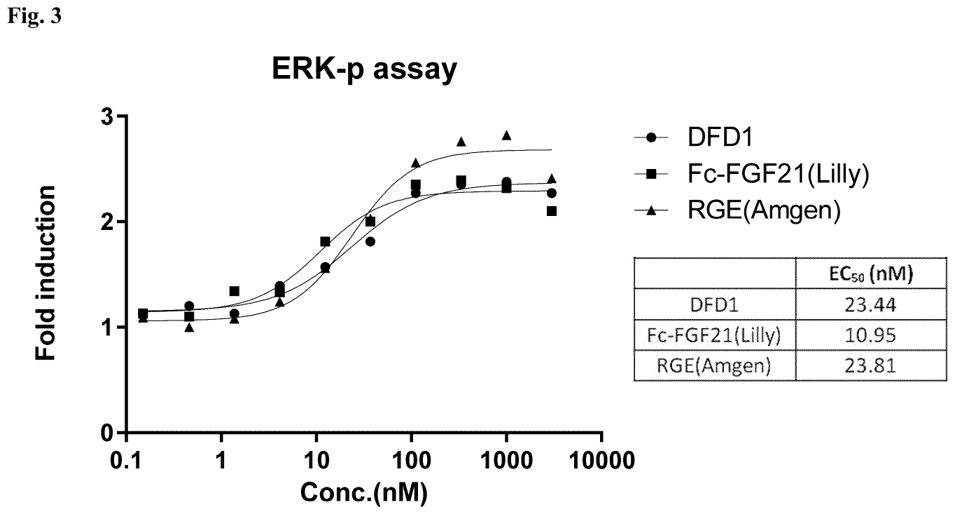

Specifically, the FGF21 activities of the fusion proteins were measured by using the concentrates containing the fusion proteins prepared in Preparation Example 1-5 and the control proteins in accordance with the methods described in Experimental Example 1-1. The results are shown in FIG. 3.

It was confirmed that DFD1 and RGE (Amgen) had similar in vitro activity, while Fc-FGF21 (Lilly) had in vitro activity two times higher than those of the other proteins, as shown in FIG. 3.

### Experimental Example 2. Evaluation of Stability of Fusion Proteins

### Experimental Example 2-1. Experimental Method for Evaluating Stability

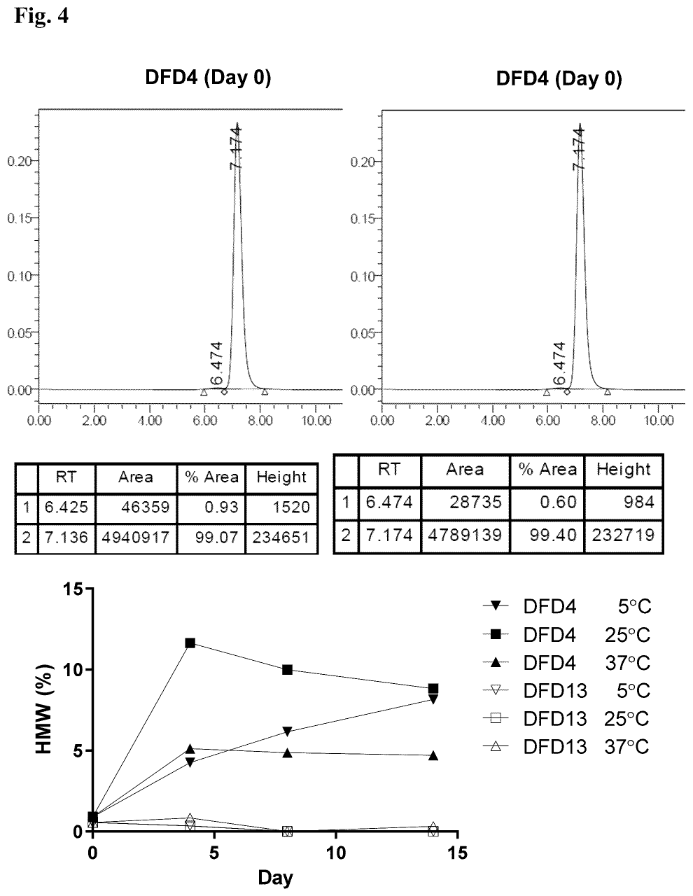

In order to measure the quantity of protein aggregates at the initial stage of the sample preparation, high molecular weight aggregates (% HMW) were quantified using a size-exclusion chromatography (SEC-HPLC) method. The results are shown in FIG. 4.

Specifically, a TOSOHAAS™ model TSK-GEL® G3000SW_{XL} column was used for the SEC-HPLC method. The column was equilibrated by flowing a buffer solution (1×PBS, 1 mM EDTA, pH 7.4) at a flow rate of 1 mL/min. The DFD4 and DFD13 protein stock solutions prepared in Preparation Examples 1-5 were concentrated to a target concentration of 20 mg/mL or higher at 3,000 rpm using a 30,000 MW cut-off centrifugation filter at 4° C. After the measurement of the concentration of each sample by BCA quantitative analysis, the samples were diluted with a buffer solution (1×PBS, 1 mM EDTA, pH 7.4) to a final concentration of 20 mg/mL. In order to measure the initial % HMW of DED4 and DFD13, 20 mg/mL of the samples were diluted with the buffer solution (1×PBS, 1 mM EDTA, pH 7.4) to a final concentration of 1 mg/mL, and each sample in a volume of 100 μL was analyzed by SEC-HPLC column.

For the stability evaluation of each sample, % HMW of the samples was measured using the SEC-HPLC method on the 4^{th}, the 8^{th} and the 14^{th} days while storing them at 5° C., 25° C. and 37° C. for two weeks.

As shown in FIG. 4, it was confirmed that DFD13 had a lower quantity of high molecular weight aggregates (HMW %) at the initial stage and up to the point of 2 weeks as compared with DFD4, indicating that the introduction of the EIRP (SEQ ID NO: 68) mutation improves the stability of the FGF21 mutant fusion protein, thereby reducing HMW % significantly.

### Experimental Example 2-2. Stability Results

In order to investigate the effects of the EIRP (SEQ ID NO: 68) mutation introduced into the original sequence LLLE (SEQ ID NO: 81) (amino acid residues at 98-101 of SEQ ID NO: 1) of FGF21 on stability, the stability of DFD4 (SEQ ID NO: 29) and DFD13 (SEQ ID NO: 35) was measured in accordance with the methods described in Experimental Example 2-1. The analysis results for the zero-hour sample (initial stage; Day 0) and 4-, 8-, and 14 day-stored samples of DFD4 and DFD13 are summarized in Table 4 below (in Table 4, N.D. means “not detected”).

TABLE 4
Stability of DFD4 and DFD13 for 2 weeks at a
concentration of 20 mg/mL (% HMW)

| | DFD4 | | | DFD13 | | |
|---|---|---|---|---|---|---|
| Day | 5° C. | 25° C. | 37° C. | 5° C. | 25° C. | 37° C. |
| 0 | 0.91 | | | 0.56 | | |
| 4 | 4.25 | 11.64 | 5.12 | 0.36 | 0.34 | 0.84 |
| 8 | 6.16 | 9.99 | 4.87 | N.D. | N.D. | N.D. |
| 14 | 8.15 | 8.83 | 4.71 | N.D. | N.D. | 0.32 |

As shown in Table 4, the quantity of % HMW at the initial stage (Day 0) was 0.91% for DFD4, and 0.56% for DFD13. After 2 weeks, the amount of % HMW increased to 8.83% for DFD4, but it was not observed in DFD13, under the condition of storage at 25° C. DFD13 was shown to have a lower % HMW rate at the initial stage and 2 weeks, as compared with DFD4, which indicates that the % HMW rate of FGF21 mutant fusion protein decreased significantly due to the introduction of the EIRP (SEQ ID NO: 68) mutation.

### Experimental Example 3. Pharmacokinetic Assessment of Fusion Proteins

### Experimental Example 3-1. Experimental Method for Pharmacokinetic Assessment

Six-week old male ICR mice purchased from Orient BIO (Korea) were partitioned into groups (n=3/blood sampling time) in order to have similar mean values for body weight one day before drug treatment, and subcutaneously administered once with a respective sample at 1 mg/kg (2 mg/kg for RGE). Blood samples were then collected at 1, 4, 8, 12, 24, 48, 72, and 96 hours after the injection, respectively. The concentration of intact full length FGF21 protein in the blood was measured using a Intact human FGF21 ELISA Kit (F1231-K01, Eagle Biosciences, USA), which has immunoreactivity to the N-terminus and C-terminus of FGF21 protein. The concentrations of the samples in the blood collected until 96 hours after the subcutaneous injection of each fusion protein into the mice were measured, and pharmacokinetic parameters of each sample were calculated.

### Experimental Example 3-2. Assessment of Pharmacokinetic Activity

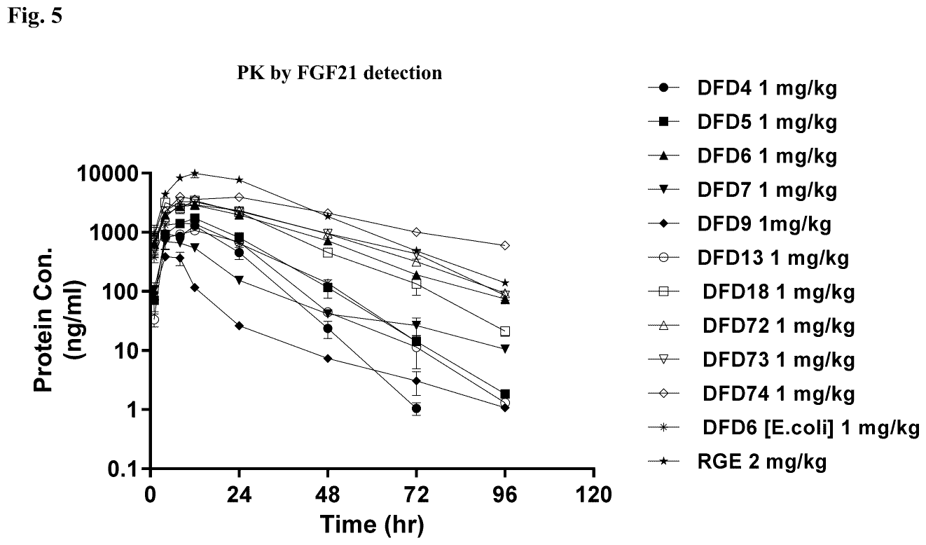

Based on the graph showing the concentrations of each protein in the blood versus time after the subcutaneous administration of fusion proteins in mice (FIG. 5), the pharmacokinetic parameters were calculated. The data are shown in Table 5 below.

TABLE 5

| | | | | | | | | | | | DFD6 | |
|---|---|---|---|---|---|---|---|---|---|---|---|---|
| Parameters | DFD4 | DFD5 | DFD6 | DFD7 | DFD9 | DFD13 | DFD18 | DFD72 | DFD73 | DFD74 | (E.coli) | RGE* |
| Tₘₐₓ (hour) | 12 | 12 | 12 | 4 | 4 | 12 | 12 | 8 | 8 | 8 | 8 | 12 |
| Cₘₐₓ (ng/mL) | 1288 | 1732 | 2868 | 696 | 384 | 1070 | 3428 | 2962 | 3296 | 3996 | 1399 | 9921 |
| AUCₗₐₛₜ | 25856 | 40706 | 100107 | 14118 | 4656 | 28785 | 104230 | 115977 | 123511 | 206634 | 37269 | 325747 |
| (ng · hr/mL) | | | | | | | | | | | | |
| Half-life (hour) | 5.5 | 8.0 | 14.9 | 19.7 | 17.4 | 7.1 | 11.0 | 14.4 | 16.6 | 26.0 | 9.1 | 12.9 |

The pharmacokinetic profile of each fusion protein was compared and evaluated based on the value of the area under the curve (AUC) indicating the degree of drug exposure.

As shown in Table 5, upon comparing DFD4 with DFD13, and DFD6 with DFD73, it was determined that the introduction of the EIRP (SEQ ID NO: 68) sequence resulted in an approximate 10 to 20% increase in AUC value. Comparing DFD9 with DFD4, the introduction of TGLEAV (SEQ ID NO: 69) resulted in an approximate 6-fold increase in AUC value.

Furthermore, the mutations of TGLEAN (SEQ ID NO: 70), G170N and G174N are designed to extend the half-life by introducing N-glycosylation into the C-terminus of FGF21, which is known to be proteolyzed in vivo. The increase in AUC due to the introduction of N-glycosylation was confirmed by comparing the mutants with each control material. In order to confirm the effect of improvement in AUC due to the introduction of N-glycosylation, the AUC value for DFD6 (E. coli) produced by E. coli which has no glycosylation was compared with that in DFD6 produced by a human cell line. DFD6 produced by the human cell line showed a 3-fold or higher increase in the AUC value as compared with DFD6 (E. coli) produced by E. coli, which demonstrated an improvement of pharmacokinetic profile due to glycosylation.

The A180E is a mutation disclosed in WO 2009/149171 owned by Amgen Inc. When the mutation of A180E was further introduced into the mutant DFD13 or DFD73 including the mutation of TGLEAV (SEQ ID NO: 69) or G170N, respectively, the resulting mutant DFD18 or DFD74, respectively, showed an approximate 2- to 3-fold additional increase in AUC value.

In summary, it was confirmed that the pharmacokinetic parameters were improved by the introduction of various mutations and combinations thereof, as compared with DFD9, the wild-type FGF21 fusion protein. The fusion protein showing the most improved AUC value was DFD74 containing the mutations of EIRP (SEQ ID NO: 68), G170N and A180E, which showed an approximate 45-fold improvement in AUC value as compared with DFD9. Furthermore, considering RGE (Amgen) at the dose of 2 mg/kg of body weight, DFD74 may have a higher degree of drug exposure as compared with RGE. The overall effects of improvement in pharmacokinetics due to the mutations are summarized in Table 6 below.

TABLE 6

| | | Control | |
|---|---|---|---|
| | | Material | |
| | Position | vs | Assessment of |
| Mutation | of | improved | pharmacokinetic |
| sequence | mutation | material | parameters |
| EIRP | 98-101 | DFD4 vs | Improvement |
| (SEQ ID | | DFD13 | of AUC |
| NO: 68) | | DFD6 vs | |
| | | DFD73 | |
| TGLEAV | 170-174 | DFD9 vs | Improvement |
| (SEQ ID | | DFD4 | of AUC |
| NO: 69) | | | |
| TGLEAN | 170-174 | DFD9 vs | Improvement |
| (SEQ ID | | DFD5 | of AUC |
| NO: 70) | | | |
| G170N | 170 | DFD9 vs | Improvement |
| | | DFD6 | of AUC |
| | | DFD6 | Improvement |
| | | (E. coli) | of AUC |
| | | vs | |
| | | DFD6 | |
| G174N | 174 | DFD9 vs | Improvement |
| | | DFD7 | of AUC |
| AI80E | 180 | DFD13 vs | Improvement |
| | | DFD18 | of AUC |
| | | DFD73 vs | Improvement |
| | | DFD74 | of AUC |

### Experimental Example 4. Activity Evaluation of Fusion Proteins in Ob/Ob Mice

### Experimental Example 4-1. Experimental Method for Evaluating Activity in Ob/Ob Mice

The ob/ob mice, characterized as exhibiting hyperglycemia, insulin resistance, hyperphagia, fatty liver and obesity due to a genetic deficiency in leptin, are widely used for the study of type 2 diabetes. Male ob/ob mice (Harlan, USA) were purchased from Raonbio (Korea). These mice were 5 to 6 weeks old at the time of arrival, and 8 to 9 weeks old at the time of drug treatment after 3 weeks of adaptation. The mice were partitioned into groups (n=8/group) in order to have similar mean values for body weight and caudal blood glucose levels one day before the drug treatment (Day 0), and the samples were subcutaneously administered once according to each of their respective dosages. Dulbecco's phosphate buffered saline (DPBS, Gibco, USA) was administered as the vehicle treatment, and the glucose concentration in the blood was measured using a glucose meter, GLUCODR™ (All Medicus, Korea). The non-fasting glucose levels and body weights were measured every day until the 14^{th} day after administration. Glycated hemoglobin levels were also measured in each group before the administration and after the test. The glycated hemoglobin levels were calculated using a DCA™ 2000 HbA1c kit (Siemens, 5035C).

### Experimental Example 4-2. Evaluation of Activity in Ob/Ob Mice

The changes in non-fasting blood glucose levels and body weights in male ob/ob mice were observed after single subcutaneous injection of 30 or 100 nmol/kg of DFD18 and DFD72, or 10, 30 or 100 nmol/kg of DFD74.

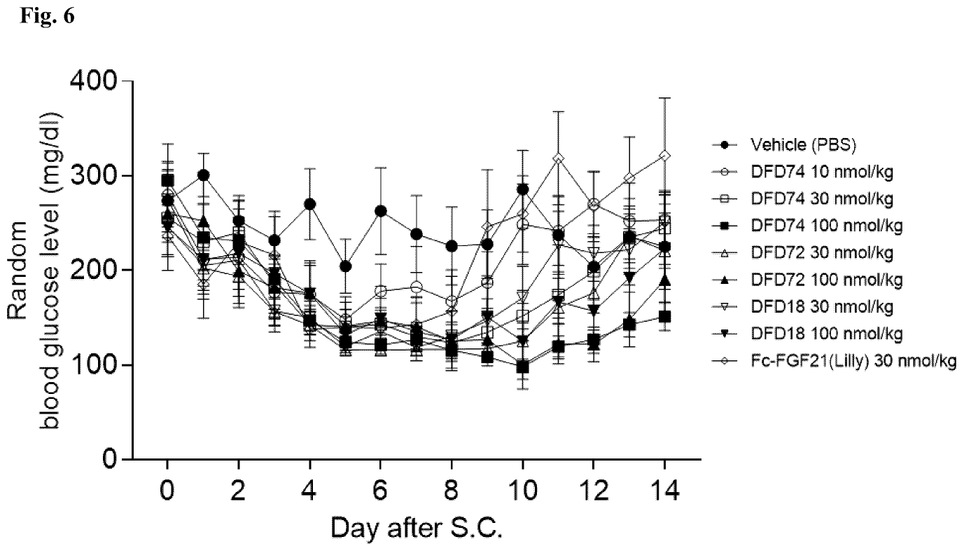

It was confirmed that DFD18, DFD72 and DFD74 all had the effect of lowering blood glucose level in a dose-dependent manner. Comparing the three agents at the high dose of 100 nmol/kg, DFD72 and DFD74 showed an improved effect on lowering blood glucose level than DED18 (FIG. 6). In addition, Fc-FGF21 (Lilly) which was used as a control material in the test, was less effective in lowing blood glucose level as compared with DFD18, DFD72 and DFD74 at the same dose level (30 nmol/kg).

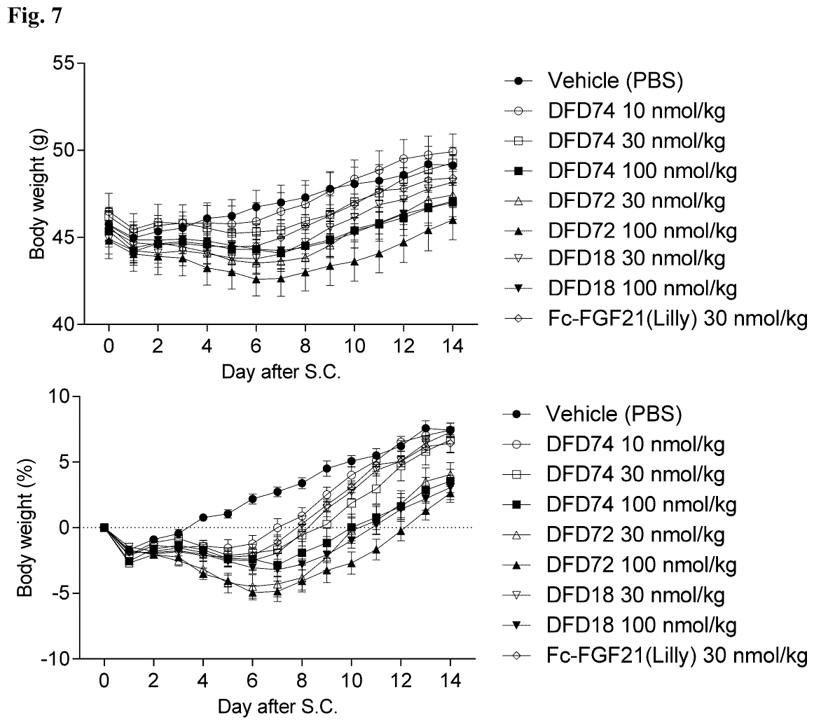

As for the effect on body weight reduction, comparing the three agents at the high dose of 100 nmol/kg, DFD72 was the most effective in ob/ob mice resulting in an approximate 6% reduction in body weight, and DFD18 was the next most effective, followed by DFD74 (FIG. 7).

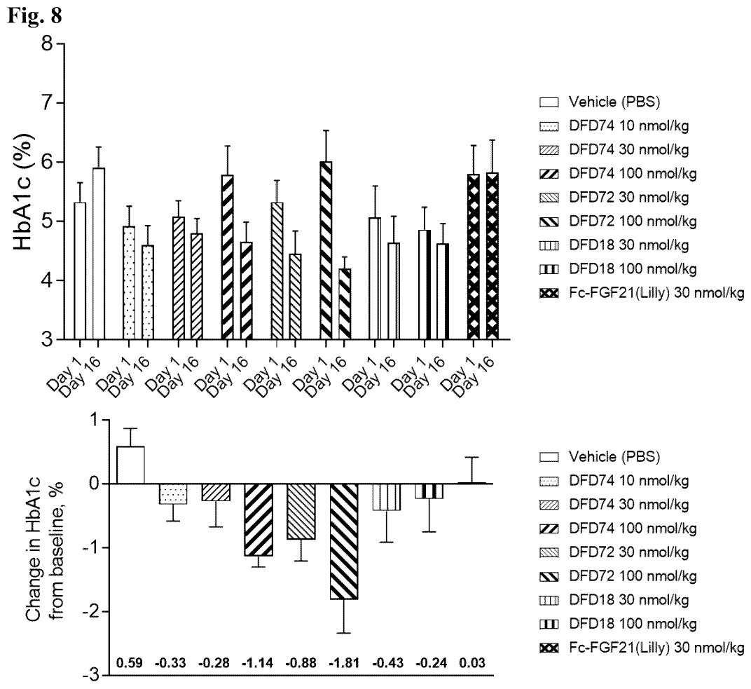

After the termination of the test, the glycated hemoglobin levels indicative of the mean values of blood glucose were measured and the changes in mean blood glucose were analyzed in each test group. All of the treated groups except the control group treated with control protein Fc-FGF21 (Lilly) showed negative values in the differences between before administration and after the test, which confirmed the effectiveness of the test proteins as compared with the control material in lowering blood glucose (FIG. 8).

### Experimental Example 5. Activity Evaluation of Fusion Proteins in HFD/STZ Mice

### Experimental Example 5-1. Experimental Method for Evaluating Activity in HFD/STZ Mice

The effects of the FGF21 mutant fusion proteins on lowering blood glucose and body weight were compared and evaluated in another diabetic model, the HFD/STZ mouse model. Conventional dietary-induced obesity mouse models (induced by feeding 60 kcal % high fat diet to C57BL/6 mice for eight weeks or longer) have weak hyperglycemic and diabetic features, although they invoke insulin resistance. The HFD/STZ mice, which may compensate for defects in the conventional dietary-induced obesity mouse models, are capable of producing dysfunctional β cells in the pancreas and decreased secretion of insulin as a result of a high fat diet (HFD) and administration of low level streptozotocin (STZ), and are therefore useful for pharmacological studies of type 2 diabetes.

Specifically, in order to induce the HFD/STZ mouse model, C57BL/6 mice (Japan SLC) were fed on a 60 kcal % high fat diet for four weeks, and then 50 mg/kg of STZ (Sigma, 85882) was administered intraperitoneally every day for 3 days to induce dysfunction in the B cells of the pancreas. After feeding on the high fat diet for an additional 2 weeks, the mice with non-fasting blood glucose levels of 200 mg/dL or higher were used for the test. The mice were partitioned into groups (n=6/group) in order to have similar mean values of body weight and caudal blood glucose levels one day before the drug treatment (Day 0), and the samples were subcutaneously administered once according to each of their respective dosages. Dulbecco's phosphate buffered saline (DPBS, Gibco, USA) was administered as the vehicle treatment, and the glucose concentration in the blood was measured using a glucose meter, GLUCODR™ (All Medicus, Korea). The non-fasting glucose levels and body weights were measured every day until the 14^{th} day after administration. Glycated hemoglobin levels were also measured in each group before the administration and after the test. The glycated hemoglobin levels were calculated using a DCA™ 2000 HbA1c kit (Siemens, 5035C).

### Experimental Example 5-2. Activity Evaluation in HFD/STZ Mice

The changes in non-fasting blood glucose levels and body weights over time in male HFD/STZ mice were observed after single subcutaneous injection of 10 nmol/kg of DFD72 or DFD74.

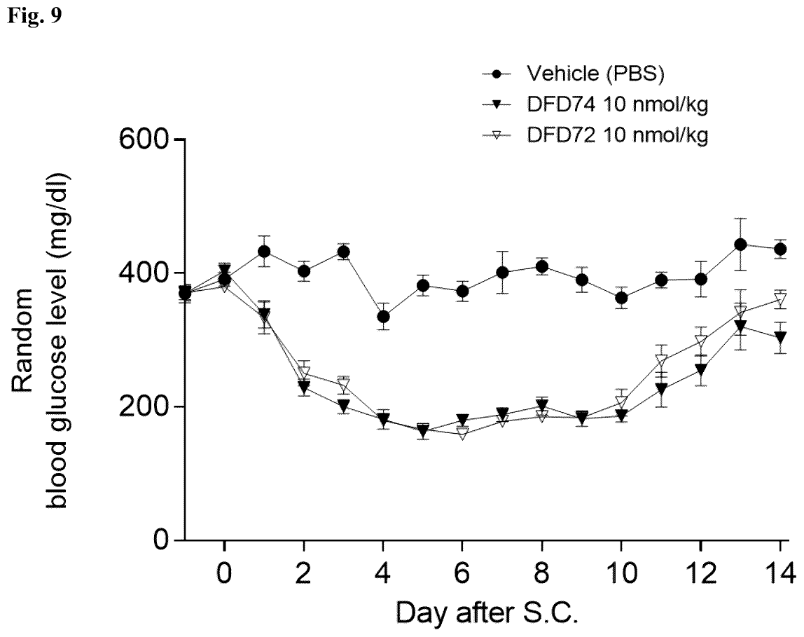

Regarding the changes in non-fasting blood glucose levels, it was confirmed that DFD72 and DFD74 had similar effects on lowering blood glucose levels, and the blood glucose lowering effect was maintained until the 10^{th} day after administration and then lost with metabolism of the drugs after the 10^{th} day (FIG. 9). DFD72 showed a more prolonged effect than DFD74 in terms of changes in non-fasting blood glucose levels after the 10^{th} day after administration.

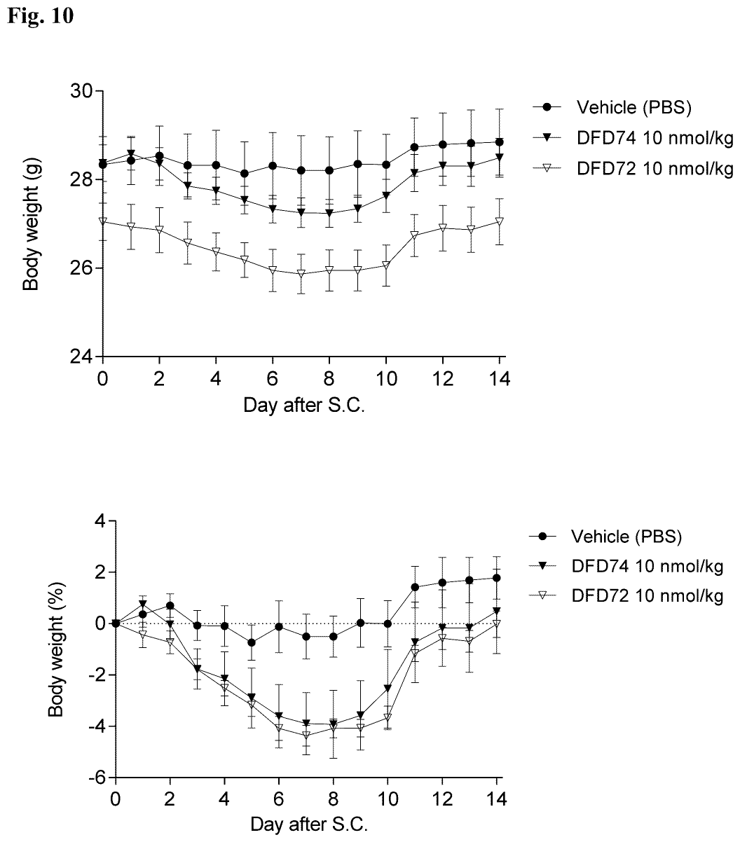

In terms of the effect on body weight reduction due to the administration of FGF21 mutant proteins, it was confirmed that both DFD72 and DFD74 had similar effects on reducing body weight by approximately 5%, and the effect disappeared after the 10^{th} day after administration (FIG. 10).

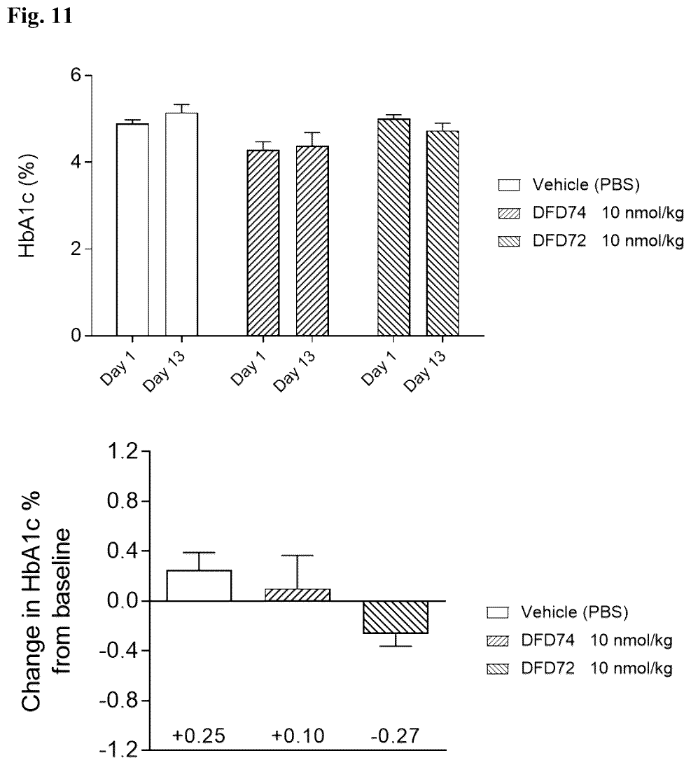

After the termination of the test, the glycated hemoglobin levels indicative of the mean value of blood glucose were measured and the changes in mean blood glucose were analyzed in each test group. While the vehicle group had an increase of 0.25 in glycated hemoglobin levels, the group treated with DFD74 had an increase of 0.1 and the group treated with DFD72 had an decrease of 0.27 (FIG. 11).

### Experimental Example 6. Activity of Fusion Proteins in Diet-Induced Obese Mice

### Experimental Example 6-1. Experimental Method for Evaluating Activities in Diet-Induced Obese Mice

The body weight-reduction effect of DFD18, an FGF21 mutant fusion protein, was evaluated in diet-induced obese mice. For the diet-induced obesity model, C57BL/6J mice were purchased from Central Lab. Animal Inc. and fed on a high-fat diet containing 60 kcal % fat (Research diet) for 8 to 12 weeks. The mice were partitioned into groups (n=8/group) in order to have a similar mean value of body weight one day before the drug treatment (Day 0), and then 30 nmol/kg of samples were subcutaneously administered once. The changes in body weights were compared with the group treated with vehicle (PBS).

### Experimental Example 6-2. Protein Activity in Diet-Induced Obese Mice

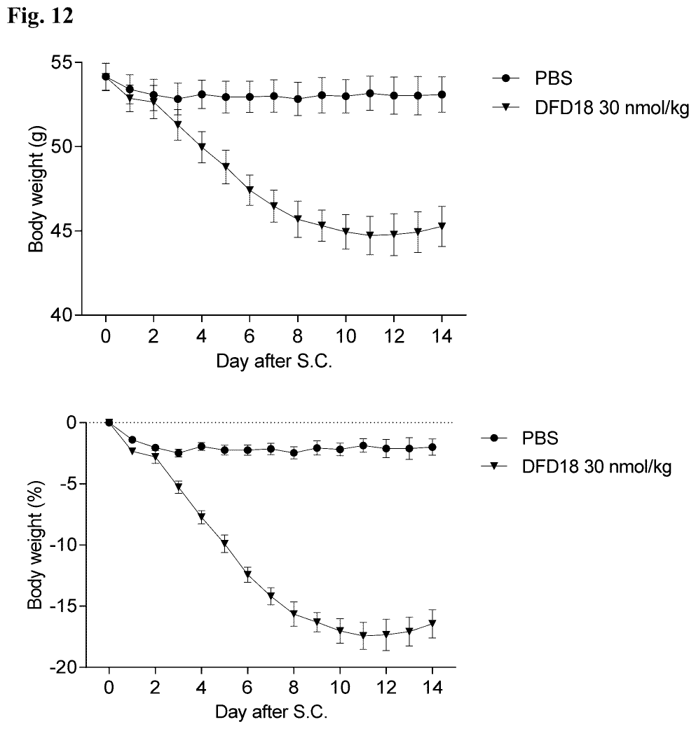

For changes in body weight over time in the diet-induced obesity mouse model following single administration of 30 nmol/kg DFD18, it was confirmed that the weight-reducing effect was continuing by the 10^{th} day after the administration, and the maximum weight reduction (about 18%) was at the 11^{th} day after the administration, which was maintained by the 14^{th} day (FIG. 12).

### Preparation Example 2. Preparation and Purification of Dual Function Proteins

### Preparation Example 2-1. Preparation of Expression Vectors for Expression of Dual Function Proteins

In order to identify the effects of the sequence of the GLP-1 mutant protein and the sequence of the Fc hinge fused thereto on the in vitro activity, pharmacokinetic profiles and pharmacological efficacy, various sequences for the Fc-fused GLP-1 mutant proteins were designed. The sequences of the GLP-1 mutant proteins are listed in Table 7 below, and the sequences of Fc-fused GLP-1 mutants are listed in Table 8.

TABLE 7

| SEQ ID NO: | Sequence of GLP-1 mutant protein |
|---|---|
| 43 | GLP-1(A2G) |
| 44 | GLP-1(GE) |
| 45 | GLP-1(GG) |
| 46 | GLP-1(GEG) |

TABLE 8

| SEQ ID NO: | Fc-fused GLP-1 mutant protein |
|---|---|
| 49 | DFD52: GLP1(A2G)-HyFc5 |
| 50 | DFD53: GLP1(A2G)-HyFc40 |
| 51 | DFD54: GLP1(GE)-HyFc5 |
| 52 | DFD55: GLP1(GE)-HyFc40 |
| 53 | DFD56: GLP1(GG)-HyFc5 |
| 54 | DFD57: GLP1(GG)-HyFc40 |
| 55 | DFD58: GLP1(GEG)-HyFc5 |
| 56 | DFD59: GLP1(GEG)-HyFc40 |

In Table 8, HyFc5 refers to SEQ ID NO: 47, and HyFc40 refers to SEQ ID NO: 48.

In order to investigate the effects of the sequences of the GLP-1 mutant proteins and FGF21 mutant proteins, the sequence of the Fe hinge fused to the GLP-1 mutants, the sequence of the linker connected between the FGF21 mutant proteins and Fc on the in vitro activity, pharmacokinetic profiles and pharmacological efficacy, various sequences for the dual function proteins were designed. The sequences of the dual function proteins including the GLP-1 mutant proteins and FGF21 mutant proteins are listed in Table 9 below. Each dual function protein contains a GLP-1 mutant protein, an Fc region of an immunoglobulin, a linker and an FGF21 mutant protein connected in this order from the N-terminus to C-terminus.

TABLE 9

| | | Sequence | | | |
|---|---|---|---|---|---|
| | | of | | | Changes |
| SEQ | | GLP-1 | | | In |
| ID | Material | mutant | Fusion | Linker | FGF21 |
| NO: | code | protein | carrier | sequence | sequence |
| 58 | DFD23 | GLP-1 | hyFc40 | GS3 | FGF21 |
| | | (A2G) | (SEQ ID | (SEQ ID | (EIRP |
| | | | NO: | NO: 4) | (SEQ ID |
| | | | 48) | | NO: 68), |
| | | | | | TGLEAV |
| | | | | | (SEQ ID |
| | | | | | NO: |
| | | | | | 69)) |
| 59 | DFD24 | GLP-1 | hyFc5 | GS3 | FGF21 |
| | | (GE) | (SEQ ID | (SEQ ID | (EIRP |
| | | | NO: 47) | NO: 4) | (SEQ ID |
| | | | | | NO: 68), |
| | | | | | TGLEAV |
| | | | | | (SEQ ID |
| | | | | | NO: |
| | | | | | 69)) |
| 60 | DFD25 | GLP-1 | hyFc40 | GS3 | FGF21 |
| | | (GE) | (SEQ ID | (SEQ ID | (EIRP |
| | | | NO: 48) | NO: 4) | (SEQ ID |
| | | | | | NO: 68), |
| | | | | | TGLEAV |
| | | | | | (SEQ |
| | | | | | ID NO: |
| | | | | | 69)) |
| 61 | DFD26 | GLP-1 | hyFc5 | GS3 | FGF21 |
| | | (GG) | (SEQ ID | (SEQ ID | (EIRP |
| | | | NO: 47) | NO: 4) | (SEQ ID |
| | | | | | NO: 68), |
| | | | | | TGLEAV |
| | | | | | (SEQ ID |
| | | | | | NO: |
| | | | | | 69)) |
| 62 | DFD27 | GLP-1 | hyFc40 | GS3 | FGF21 |
| | | (GG) | (SEQ ID | (SEQ ID | (EIRP |
| | | | NO: 48) | NO: 4) | (SEQ ID |
| | | | | | NO: 68), |
| | | | | | TGLEAV |
| | | | | | (SEQ ID |
| | | | | | NO: |
| | | | | | 69)) |
| 63 | DFD28 | GLP-1 | hyFc5 | GS3 | FGF21 |
| | | (GEG) | (SEQ ID | (SEQ ID | (EIRP |
| | | | NO: 47) | NO: 4) | (SEQ ID |
| | | | | | NO: 68), |
| | | | | | TGLEAV |
| | | | | | (SEQ ID |
| | | | | | NO: |
| | | | | | 69)) |
| 64 | DFD29 | GLP-1 | hyFc40 | GS3 | FGF21 |
| | | (GEG) | (SEQ ID | (SEQ ID | (EIRP, |
| | | | NO: 48) | NO: 4) | TGLEAV) |
| 65 | DFD69 | GLP-1 | hyFc40 | GS3 | FGF21 |
| | | (GEG) | (SEQ ID | (SEQ ID | (EIRP |
| | | | NO: 48) | NO: 4) | (SEQ ID |
| | | | | | NO: 68), |
| | | | | | TGLEAV |
| | | | | | (SEQ ID |
| | | | | | NO: |
| | | | | | 69)) |
| | | | | | AI80E) |
| 66 | DFD112 | GLP-1 | hyFc40 | GS3 | FGF21 |
| | | (GEG) | (SEQ ID | (SEQ ID | (EIRP |
| | | | NO: 48) | NO: 4) | (SEQ ID |
| | | | | | NO: 68, |
| | | | | | TGLEAN |
| | | | | | (SEQ |
| | | | | | ID NO: |
| | | | | | 70), |
| | | | | | A180E) |
| 67 | DFD114 | GLP-1 | hyFc40 | GS3 | FGF21 |
| | | (GEG) | (SEQ ID | (SEQ ID | (EIRP |
| | | | NO: 48) | NO: 4) | (SEQ ID |
| | | | | | NO: 68), |
| | | | | | G170N, |
| | | | | | A180E) |

Specifically, the nucleotide sequences encoding each of the dual function proteins were synthesized after consulting with Bioneer Corporation (Korea) based on the amino acid sequence of each protein. NheI and NotI restriction enzyme sequences were added to the 5′ terminus and 3′ terminus of the nucleotide sequences encoding each of the dual function proteins and an initiation codon for protein translation and a leader sequence (MDAMLRGLCCVLLLCGAVFVSPSHA) (SEQ ID NO: 83) enabling secretion of the expressed protein to the outside of a cell were inserted next to the restriction enzyme sequence at the 5′ terminus. A termination codon was inserted next to the nucleotide sequence, which encodes each of the dual function proteins. The nucleotide sequence encoding each of the dual function proteins was cloned into a pTrans-empty expression vector by using the two restriction enzymes NheI and NotI. The pTrans-empty expression vector, which has a simple structure including a CMV promoter, a pUC-derived replication origin, an SV40-derived replication origin and an ampicillin-resistance gene, was purchased from CEVEC Pharmaceuticals (Germany).

### Preparation Example 2-2. Construction of Plasmid DNA for Expression of Fc-Fused GLP-1 Mutant and Dual Function Proteins

E. coli was transformed with each of the expression vectors constructed in Preparation Example 2-1 to obtain a large quantity of plasmid DNA to be used for expression. E. coli cells, with cell walls weakened through heat shock, were transformed with each expression vector, and the transformants were plated out on an LB plate to obtain colonies. The colonies thus obtained were inoculated into LB media, cultured at 37° C. for 16 hours, and each E. coli culture containing each expression vector was obtained in a volume of 100 mL. The E. coli thereafter obtained was centrifuged to remove the culture medium, and then P1, P2, P3 solutions (QIAGEN, Cat No.: 12963) were added to break the cell walls, thereby obtaining a DNA suspension in which proteins and DNA were separated. Plasmid DNA was purified from the DNA suspension thus obtained by using a QIAGEN™ DNA purification column. The eluted plasmid DNA was identified by agarose gel electrophoresis, and the concentrations and purities were measured using a NANODROP™ device (Thermo Scientific, NANODROP™ Lite). The DNA thus obtained was used for expression.

### Preparation Example 2-3. Expression of Fc-Fused GLP-1 Mutants and Dual Function Proteins in CAP-T Cells

Human cell lines were transformed with each plasmid DNA obtained in Preparation Example 2-2. Each plasmid DNA type was transduced into CAP-T cells (CEVEC), which had been cultured in PEM medium (Life Technologies), by using a PEI solution (Polyplus, Cat. No.: 101-10N). The mixed solution of DNA and the PEI solution was mixed with the cell suspension using FREESTYLE™ 293 expression medium (Invitrogen), cultured at 37° C. for 5 hours, and PEM medium was added. After culturing at 37° C. for 5-7 days, the culture was centrifuged to remove cells and supernatant containing each protein was obtained.

### Preparation Example 2-4. Purification of Fe-Fused GLP-1 Mutants and Dual Function Proteins

Protein A affinity chromatography column (GE Healthcare) was equilibrated with 1×PBS buffer solution (pH 7.4). The culture supernatant including each of the Fc-fused GLP-1 mutants and dual function proteins obtained in Preparation Example 2-3 was filtered with a 0.2 μm filter, and then loaded into a Protein A affinity chromatography column. The column was washed with 1×PBS buffer solution (pH 7.4) and then the proteins were eluted using 100 mM glycine buffer solution (pH 3.0). The proteins obtained by affinity chromatography were purified using an anion exchange resin column (POROS® HQ 50 μm, Thermo Fisher Scientific). The anion exchange resin column was equilibrated with 50 mM Tris buffer solution (pH 8.0), before the proteins eluted from the affinity chromatography were loaded thereto.

After washing the column with 50 mM Tris buffer solution (pH 8.0), 50 mM Tris buffer solution (pH 8.0) was dispensed along the concentration gradient and the eluted fractions were analyzed. Each eluted fraction was analyzed by using size exclusion chromatography (SEC-HPLC), and the fractions including the Fc-fused GLP-1 mutants and dual function proteins with high purity were collected and dialyzed overnight at 4° C. using a final buffer solution (1×PBS, 1 mM EDTA, pH 7.4). Upon completion of the dialysis, the obtained protein stock solution was concentrated at 3,000 rpm using a 30,000 MW cut-off centrifugation filter at 4° C. The concentration of each protein was measured via BCA quantitative analysis.

### Experimental Example 7. In Vitro Activity of Dual Function Proteins

### Experimental Example 7-1. Activity of DFD23, DFD24, DFD25, DFD26, DFD27, DFD28 and DFD29

The in vitro GLP-1 activities of the dual function proteins DFD23, DFD24, DFD25, DFD26, DFD27, DFD28 and DFD29 were measured. Specifically, a CHO cell line (Eurofins, HTS163C2), overexpressing the human GLP-1 receptor was purchased and used to evaluate the GLP-1 activities of the dual function proteins. For the evaluation of activity, samples containing the fusion proteins (protein stock solutions prepared in Preparation Example 2-4; hereinafter, “sample”) were subjected to a 4-fold serial dilution at a concentration of 25 nM. After the human GLP-1 receptor-overexpressing CHO cell line was treated for 30 minutes, the intracellular cAMP produced was measured (Cisbio, 62AM4PEB). The activity of each protein was evaluated by comparing the EC₅₀ values.

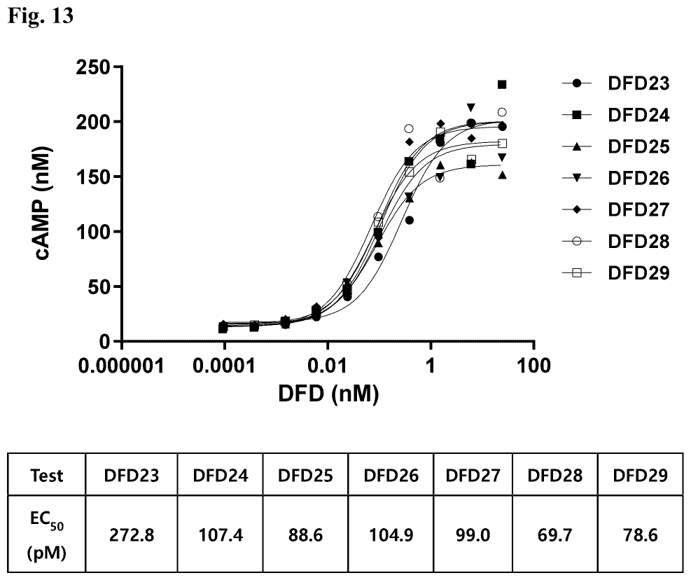

As shown in FIG. 13, the dual function protein containing the GLP-1 (A2G) sequence showed activity approximately 2-3 times lower than that for the dual function proteins containing other GLP-1 mutant sequences. No significant difference in GLP-1 activities was observed between the dual function proteins containing the mutation sequences except the GLP-1 (A2G) sequence.

### Experimental Example 7-2. Activities of DFD59, DFD69, DFD112 and DFD114

The in vitro GLP-1 activities of the dual function proteins DFD69, DFD112 and DFD114 prepared in Preparation Example 2 and DFD59 (an Fc-fused GLP-1 mutant) were measured. Specifically, a CHO cell line (Eurofins, HTS163C2) overexpressing the human GLP-1 receptor was purchased and used to evaluate the GLP-1 activities of the dual function proteins. For the evaluation of activity, the sample containing each of the fusion proteins was subjected to a 4-fold serial dilution at a concentration of 25 nM. After the human GLP-1 receptor-overexpressing CHO cell line was treated for 30 minutes, the intracellular cAMP produced was measured (Cisbio, 62AM4PEB).

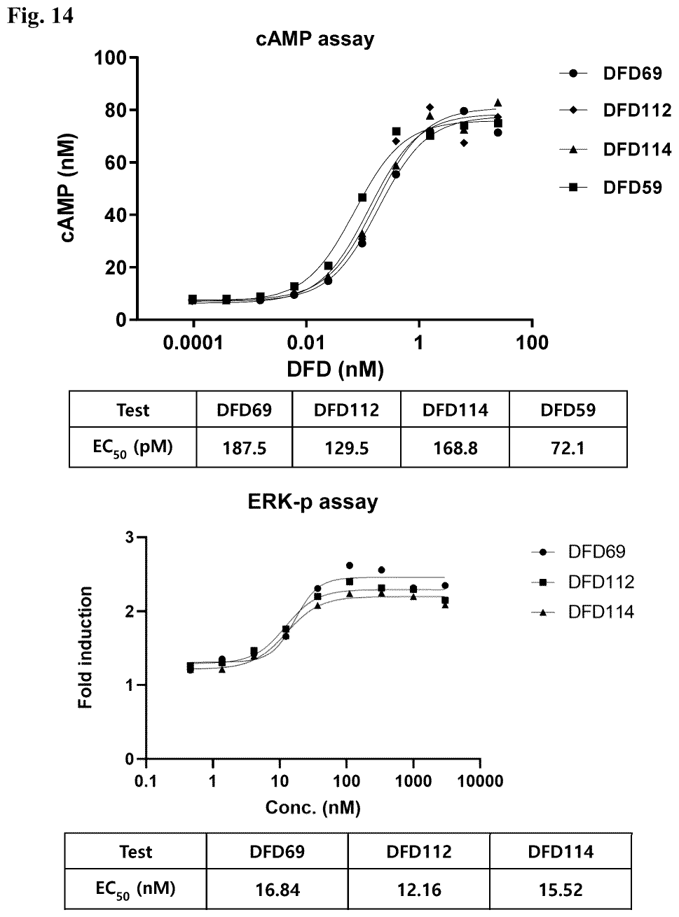

As shown in FIG. 14, the activity of each protein was evaluated by comparing the EC₅₀ value. The three dual function proteins showed similar EC₅₀ values, and DFD59 (containing no FGF21 mutant) showed activity approximately 2 times higher than that of the dual function proteins.

Next, the in vitro activities of the FGF21 portion in DFD69, DFD112 and DFD114 were measured. Specifically, the in vitro activities of the FGF21 portion in the dual function proteins were evaluated using a HEK293 cell line overexpressing human β-klotho (a co-receptor of FGF21). For the evaluation of activity, samples containing each of the dual function proteins were subjected to a 3-fold serial dilution at a concentration of 3 μM. After having been cultured in a serum-deficient state for 5 hours, the human β-klotho-overexpressing HEK293 cell line was treated for 20 minutes, before the cells were lysed by adding cytolysis buffer (Cisbio/Cat #64ERKPEG) with stirring at 60 rpm for 30 minutes at room temperature. The cell lysate solution was mixed with antibodies which can detect ERK and phosphorylated ERK, and the mixture was maintained at room temperature for 2 hours. Fluorescence was detected using a fluorometric detector (TECAN/GENiosPro). The activities were measured by comparing their EC₅₀ values.

It was confirmed that the in vitro activities of the FGF21 portion of the dual function proteins DFD69, DFD112 and DFD114 were similar, as shown in FIG. 14.

### Experimental Example 8. Pharmacokinetic Assessment of Dual Function Proteins

### Experimental Example 8-1. Experimental Method for Pharmacokinetic Assessment

Six-week old male ICR mice purchased from Orient BIO (Korea) were partitioned into groups (n=3/blood sampling time) in order to have a similar mean value of body weight one day before drug treatment, and subcutaneously administered once with a respective sample in a volume of 1 mg/kg. The blood samples were collected at 1, 4, 8, 12, 24, 48, 72, 96, 144, 192 and 240 hours after the injection, respectively. The concentration of each dual function protein in the blood was measured based on the FGF21 portion and the GLP-1-Fc portion separately. The concentration of the intact full length FGF21 portion of the dual function protein in the blood was measured using an Intact human FGF21 ELISA Kit (F1231-K01, Eagle Biosciences, USA), which has immunoreactivity to the N-terminus and C-terminus of FGF21 protein. Further, the concentration of the active GLP-1-Fc portion of the dual function protein in the blood was measured using an antibody, which has immunoreactivity to the N-terminus of GLP-1 and Fc, as determined through ELISA analysis. The concentrations of the FGF21 and GLP-1-Fc portions of each protein in the blood samples collected until 240 hours after single subcutaneous injection of each protein into the mice were measured, and the pharmacokinetic parameters of each protein was calculated.

### Experimental Example 8-2. Pharmacokinetic Activity Results

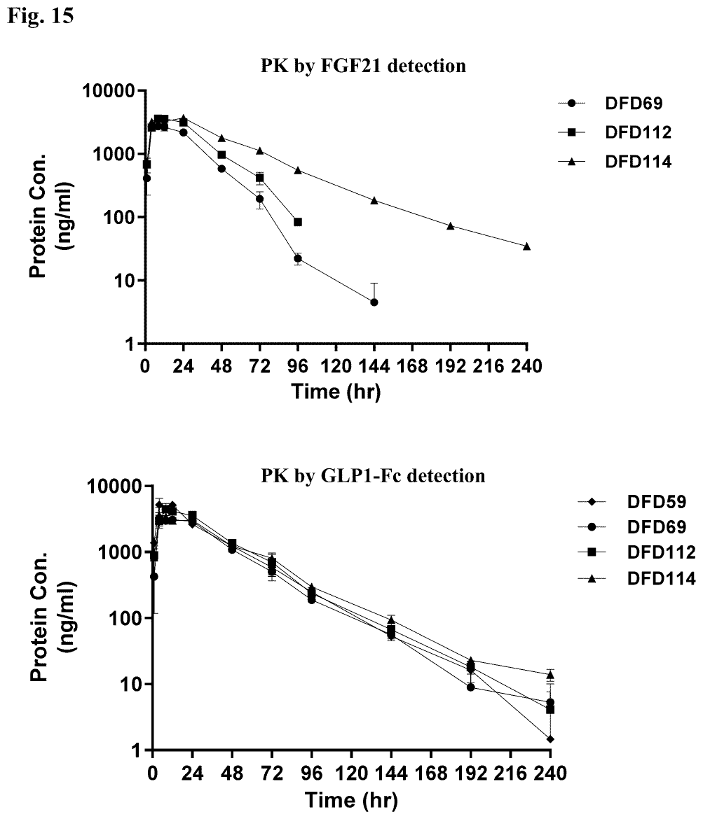

Based on the concentration of each active substance in the blood over time after single subcutaneous administration of each protein in mice (FIG. 15), pharmacokinetic parameters for the FGF21 and GLP-1-Fc portions of the dual function proteins were calculated. The data are shown in Table 10 below.

TABLE 10

| | FGF21 detection | | | GLP-1-Fc detection | | | |
|---|---|---|---|---|---|---|---|
| Parameter | DFD69 | DFD112 | DFD114 | DFD59 | DFD69 | DFD112 | DFD114 |
| Tₘₐₓ (hour) | 8 | 8 | 24 | 4 | 4 | 8 | 4 |
| Cₘₐₓ (ng/mL) | 2715 | 3619 | 3711 | 5202.1 | 3234 | 4454 | 3616 |
| AUCₗₐₛₜ (ng · hr/mL) | 100907 | 144395 | 222504 | 182852 | 149083 | 189338 | 171687 |
| Half-life (hour) | 13.4 | 14.2 | 39.9 | 20.7 | 23.3 | 24.7 | 27.2 |

The pharmacokinetic profiles of each dual function protein were compared and evaluated based on the value of the area under the curve (AUC), indicating the degree of drug exposure.

As shown in Table 10, for the pharmacokinetic parameters of the FGF21 portion, DFD114 showed the highest degree of drug exposure (AUC) and half-life, and DFD112 showed the next highest AUC value, followed by DFD69. DFD114 exhibited an approximate 2-fold or higher increase in AUC value as compared with DFD69. For the pharmacokinetics of the GLP-1-Fc portion, the four proteins (DFD59, DFD69, DFD112 and DFD114) containing the same GLP-1 mutant sequence showed similar AUC values.

### Experimental Example 9. Activity Evaluation in db/db Mice

### Experimental Example 9-1. Method for Evaluating Activities in db/db Mice

The db/db mice, characterized as having hyperglycemia, insulin resistance, hyperphagia, fatty liver and obesity due to a genetic deficiency for the leptin receptor and exhibiting more serious hyperglycemia and obesity than ob/ob mice, are widely used for the study of type 2 diabetes. Male db/db mice (Harlan, USA) were purchased from Raonbio (Korea). These mice were 5 to 6 weeks old at the time of arrival, and 8 to 9 weeks old at the time of drug treatment, after 3 weeks of adaptation. The mice were partitioned into groups (n=6/group) in order to have a similar mean value of body weight and caudal blood glucose levels one day before the drug treatment (Day 0), and the samples were subcutaneously administered once according to each of their respective dosages. Dulbecco's phosphate buffered saline (DPBS, Gibco, USA) was administered as the vehicle treatment, and the glucose concentration in the blood was measured using a glucose meter, GLUCODR™ (All Medicus, Korea). The non-fasting glucose levels and body weights were measured every day until the 14^{th} day after administration. Glycated hemoglobin levels were also measured in each group before the administration and after the test. The glycated hemoglobin levels were calculated using a DCA™ 2000 HbA1c kit (Siemens, 5035C).

### Experimental Example 9-2. Evaluation of Activity in db/db Mice

The changes in non-fasting blood glucose levels and body weights in male db/db mice were observed after single subcutaneous injection of 10 or 30 nmol/kg of dual function protein DFD114, single subcutaneous injection of 30 nmol/kg of long-acting GLP-1-Fc single function protein DFD59, and combined administration of 30 nmol/kg of DFD59 and DFD74 (which are GLP-1-Fc and Fc-FGF21 single function proteins, respectively) to compare the effect of the dual function protein DFD114 with combined administration of Fc-FGF21 and GLP-1-Fc single function proteins.

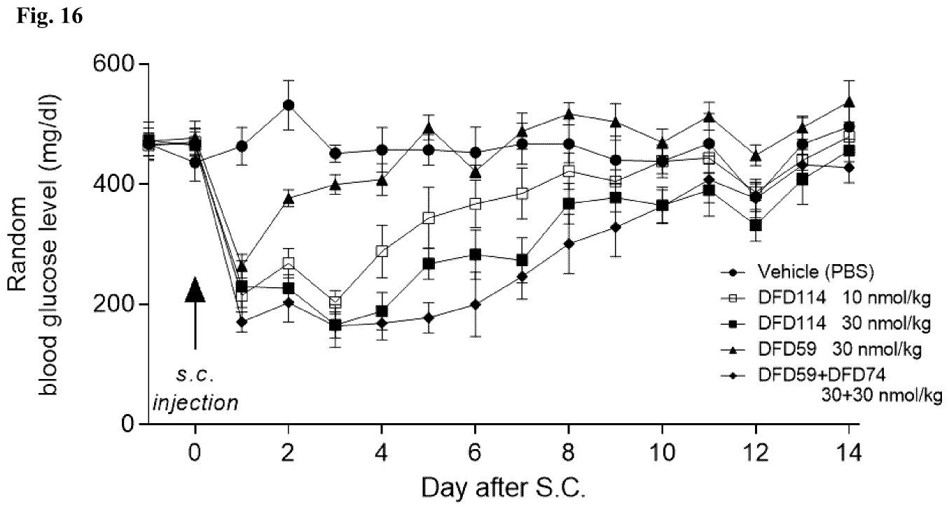

The long-acting GLP-1-Fc protein DFD59 caused a sharp reduction in blood glucose levels by the 1^{st} day after administration, but the reduction in blood glucose decreased after the 2^{nd} day and the blood glucose level was similar to that of the vehicle-treated group after the 4^{th} day. Meanwhile, the group treated with DFD114 showed excellent effects on blood glucose reduction by the 3^{rd} day after administration, and the effects on lowering blood glucose level disappeared more rapidly after the 4^{th} day from the administration at the dose of 10 nmol/kg than for 30 nmol/kg, indicating dose-dependent differences in the duration of the blood glucose lowering effect. The groups treated with combined administration of each protein showed the most sustained effects for lowering blood glucose levels as compared with those of the other groups, indicating that the combination of GLP-1 and FGF21 had an excellent effect on controlling blood glucose level (FIG. 16).

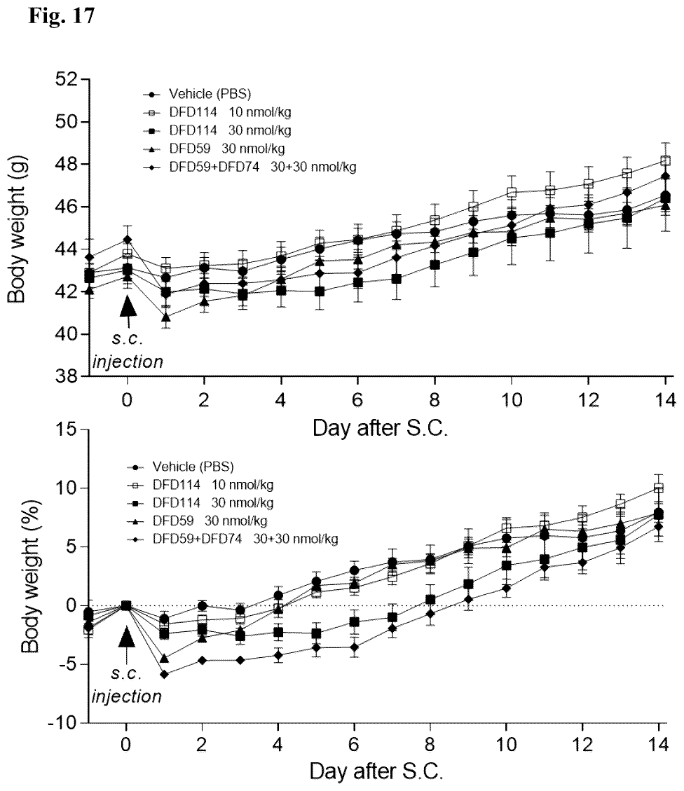

As for the effect on body weight reduction, the groups treated with a combination of DFD59 and DFD74 showed the greatest effects on reducing body weight, and the group treated with 30 nmol/kg of DFD114 also showed an outstanding effect on reducing body weight (FIG. 17).

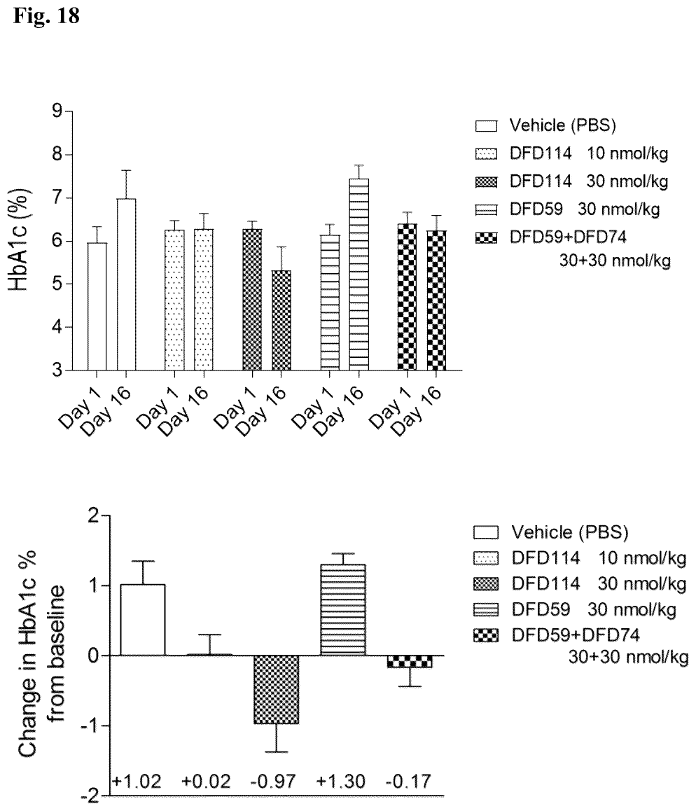

After the termination of the tests, the glycated hemoglobin levels indicative of the mean value of blood glucose were measured and the changes in mean blood glucose were analyzed in each test group. As shown in FIG. 18, the group treated with vehicle showed increased glycated hemoglobin levels after the termination of the tests as compared with the group before the administration, and the group treated with DFD59 showed a similar increase. The group treated with 30 nmol/kg of DFD114 showed the greatest decrease in glycated hemoglobin levels, and the group receiving combined administration showed the next highest effectiveness, followed by the group treated with 10 nmol/kg of DFD114. When evaluating the proteins by comparing them based on the decrease in glycated hemoglobin levels in each group treated, it was confirmed that the dual function protein DFD114 showed a stronger effect on lowering blood glucose level than GLP-1-Fc or Fc-FGF21 single function protein alone.

### Experimental Example 10. Activity of Fusion Proteins in HFD/STZ Mice

### Experimental Example 10-1. Experimental Method for Evaluating Activities in HFD/STZ Mice

The effects of the dual function proteins on lowering blood glucose and body weight were compared and evaluated in another diabetic model, the HFD/STZ mouse model.

The conventional dietary-induced obesity mouse model (induced by feeding 60 kcal % high fat diet to C57BL/6 mice for eight weeks or longer) has weak hyperglycemic and diabetic features, although invokes insulin resistance. The HFD/STZ mice, which may compensate for the deficiencies of the conventional dietary-induced obesity mouse model, are capable of generating dysfunctional β cells of the pancreas and decreased secretion of insulin following a high fat diet (HFD) and administration of low level streptozotocin (STZ), and are used for pharmacological studies of type 2 diabetes. In order to induce the HFD/STZ mouse model, C57BL/6 mice were fed on a 60 kcal % high fat diet for four weeks, and then 50 mg/kg of STZ (Sigma, 85882) was administered intraperitoneally every day for 3 days to induce dysfunction of the β cells of the pancreas. After feeding on the high fat diet for an additional 2 weeks, the mice with non-fasting blood glucose levels of 200 mg/dL or higher were selected for the test. The mice were partitioned into groups (n=6/group) in order to have a similar mean value of body weight and caudal blood glucose levels one day before the drug treatment (Day 0), and the samples were subcutaneously administered once according to each of their respective dosages. Dulbecco's phosphate buffered saline (DPBS, Gibco, USA) was administered as the vehicle treatment, and the glucose concentration in the blood was measured using a glucose meter, GLUCODR™ (All Medicus, Korea). The non-fasting glucose levels and body weights were measured every day until the 14^{th} day after administration. Glycated hemoglobin levels were also measured in each group before the administration and after the test. The glycated hemoglobin levels were calculated using a DCA™ 2000 HbA1c kit (Siemens, 5035C).

### Experimental Example 10-2. Activity in HFD/STZ Mice

The changes in non-fasting blood glucose levels and body weights over time in male HFD/STZ mice were observed after single subcutaneous injection of 3 nmol/kg or 10 nmol/kg of dual function protein DFD114, 10 nmol/kg of Fc-fused GLP-1 mutant DFD59, or 10 nmol/kg of each of the Fc-fused FGF21 mutants DFD72 and DFD74. DFD59 and DFD74 were also subcutaneously injected once at 10 nmol/kg each in order to compare the effect of combined administration of the single function proteins with that of the dual function protein.

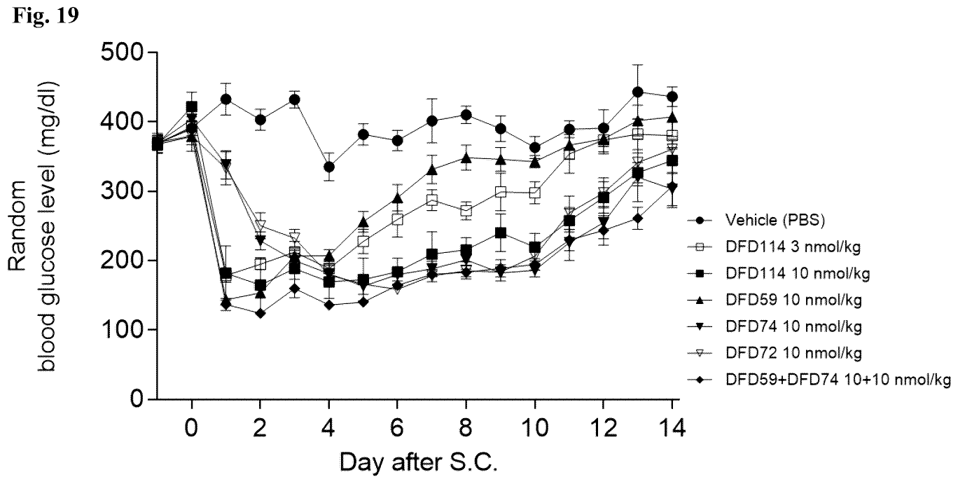

As shown in FIG. 19, regarding the changes in blood glucose levels until the 4^{th} day, DFD72 and DFD74 (long-acting FGF21 single function proteins) administration resulted in slower reductions of blood glucose, while DFD114 (long-acting protein including GLP-1), DFD59 and combined administration of DFD59 and DFD74 showed a more rapid reduction of blood glucose from the 1^{st} day of administration. Similar to the results in db/db mice, DFD59 showed a sharp reduction in blood glucose at an early stage, but the reduction of blood glucose disappeared slowly after the 4^{th} day. DFD114 showed a similar pattern at the low dose of 3 nmol/kg. In the groups treated with 10 nmol/kg of DFD114, DFD72, DFD74 and combined administration, similar non-fasting blood glucose profiles were observed.

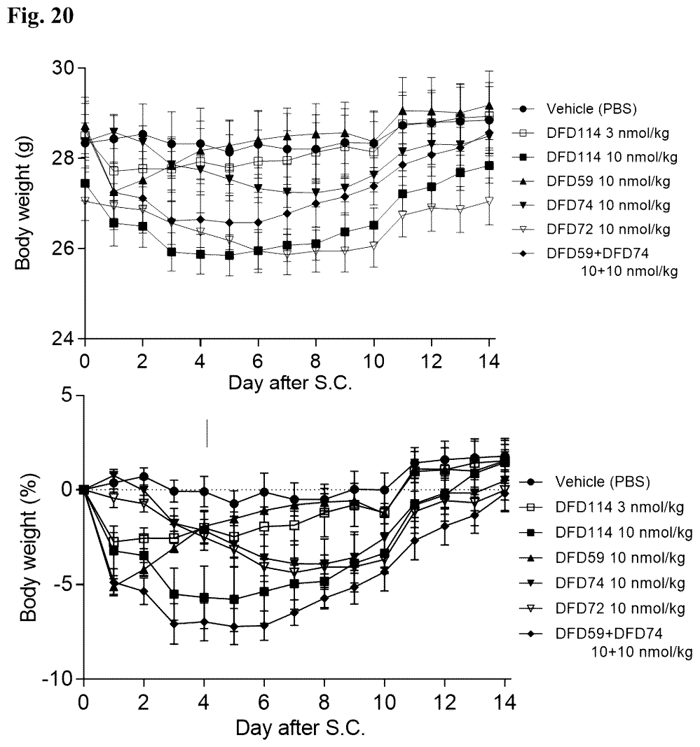

As for the effect on body weight reduction, the group treated with combined administration of DFD59 and DFD74 showed the greatest effect on body weight reduction (7 to 8%), and the group treated with 10 nmol/kg of DFD114 also showed an outstanding effect on reducing body weight (approximately 6%) (FIG. 20). The group treated with DFD59 exhibited a reduction in body weight by 5% at the 1^{st} day after administration, but the effect disappeared after the 2^{nd} day and became similar to that of the vehicle group after the 7^{th} day. The group treated with each of the long-acting FGF21 single function proteins DFD72 and DFD74 showed a slower reduction in body weight by 4 to 5% until the 7^{th} day after the administration, and the effect disappeared after the 10^{th} day.

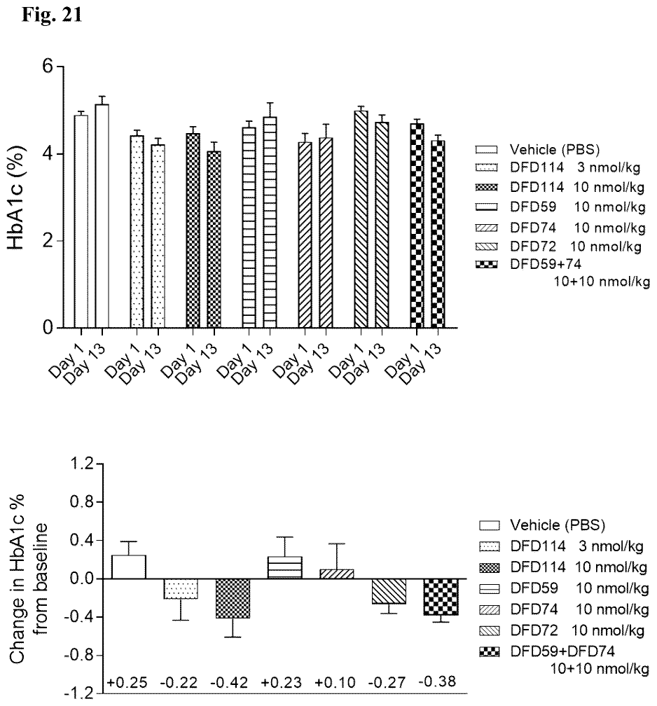

After the termination of the tests, the glycated hemoglobin levels indicative of the mean value of blood glucose were measured and the changes in mean blood glucose were analyzed in each test group (FIG. 21). The vehicle group had an increase in glycated hemoglobin levels after the termination of the test as compared with before administration, and the group treated with DFD59 showed a similar increase. In contrast, the group treated with DFD114 showed reductions in glycated hemoglobin levels in a dose-dependent manner, and the group treated with 10 nmol/kg of DFD114 had the greatest effect in terms of reduced glycated hemoglobin levels (−0.42%). The group treated with combined administration of DFD59 and DFD74 showed reduced glycated hemoglobin levels (−0.38%) similar to that of DFD114. For the long-acting FGF21 single function proteins, it was observed that DFD72 was superior to DFD74. Comparing the proteins based on the reduced levels of glycated hemoglobin in each group, it was confirmed that the dual function protein DFD114 was superior to both GLP-1-Fc and Fc-FGF21 single function proteins.

### Experimental Example 11, Prediction and Evaluation of Immunogenicity

### Experimental Example 11-1. Prediction Method for Immunogenicity and Results

In order to predict the potential immunogenicity of dual function proteins, in silico analysis of immunogenicity was performed for each protein.

Specifically, the potential immunogenicity of dual function proteins was rapidly screened by using ITOPE™ and TCED™ methods (Prediction of immunogenicity of therapeutic proteins: validity of computational tools, BioDrugs, 2010). According to the two methods, the T-cell epitope may be more accurately predicted as compared with the in silico analytical method which depends on MHC class II binding analysis only.

### Experimental Example 11-2. Ex Vivo Evaluation Method for Immunogenicity and Results

In order to evaluate the potential immunogenicity of dual function proteins, EPISCREEN™ analysis (Increased brain bio-distribution and chemical stability and decreased immunogenicity of an engineered variant of GDNF, Exp Neurol, 2015) was performed. When immunogenicity is detected, the amino acid sequences inducing immunogenicity may be identified through T-cell epitope mapping, and deimmunized mutants with minimized immunogenicity may be designed and prepared via in silico prediction to reevaluate immunogenicity.

1. A dual function protein comprising a fibroblast growth factor 21 (FGF21) mutant protein; a biologically active protein, or a mutant or fragment thereof; and an Fc region of an immunoglobulin,
wherein the FGF21 mutant protein comprises the following mutation:
(i) a substitution of the amino acid at position 180 from the N-terminus of a wild-type FGF21 protein with the amino acid E.

2. The dual function protein of claim 1, wherein the FGF21 mutant protein further comprises the following mutation:
(ii) a substitution of the amino acids at positions 170 to 174 from the N-terminus of a wild-type FGF21 protein with the amino acid sequence of TGLEAN (SEQ ID NO: 44).

3. The dual function protein of claim 1, wherein the FGF21 mutant protein further comprises the following mutation:
(iii) a substitution of the amino acid at position 170 from the N-terminus of a wild-type FGF21 protein with the amino acid N.

4. The dual function protein of claim 1, wherein the FGF21 mutant protein further comprises the following mutation:
(iv) a substitution of the amino acids at positions 98 to 101 from the N-terminus of a wild-type FGF21 protein with the amino acid sequence of EIRP (SEQ ID NO: 42);
(v) a substitution of the amino acids at positions 170 to 174 from the N-terminus of a wild-type FGF21 protein with the amino acid sequence of TGLEAV (SEQ ID NO: 43);
(vi) a substitution of the amino acid at position 174 from the N-terminus of a wild-type FGF21 protein with the amino acid N; or
(vii) a combination of the substitution (iv) and the substitution (v), or a combination of the substitution (iv) and the substitution (v).

5. The dual function protein of claim 1, wherein the amino acid residue N of the FGF21 mutant protein introduced by a mutation is glycosylated.

6. The dual function protein of claim 1, wherein the biologically active protein is one selected from the group consisting of insulin, C-peptide, leptin, glucagon, gastrin, gastric inhibitory polypeptide (GIP), amylin, calcitonin, cholecystokinin, peptide YY, neuropeptide Y, bone morphogenetic protein-6 (BMP-6), bone morphogenetic protein-9 (BMP-9), oxyntomodulin, oxytocin, glucagon-like peptide-1 (GLP-1), glucagon-like peptide-2 (GLP-2), irisin, fibronectin type III domain-containing protein 5 (FNDC5), apelin, adiponectin, C1q and tumor necrosis factor related protein (CTRP family), resistin, visfatin, omentin, retinol binding protein-4 (RBP-4), glicentin, angiopoietin, interleukin-22 (IL-22), exendin-4, growth hormone, and a combination thereof.

7. The dual function protein of claim 6, wherein the biologically active protein is one selected from GLP-1, a mutant thereof, and exendin-4.

8. The dual function protein of claim 7, wherein the mutant of GLP-1 comprises an amino acid sequence selected from the group consisting SEQ ID NOs: 43 to 46.

9. The dual function protein of claim 1, wherein the wild-type FGF21 protein comprises the amino acid sequence represented by SEQ ID NO: 1.

10. The dual function protein of claim 1, wherein the FGF21 mutant protein comprises an amino acid sequence selected from the group consisting of SEQ ID NOs: 6 to 23.

11. The dual function protein of claim 1, wherein the dual function protein further comprises a linker.

12. The dual function protein of claim 11, wherein the linker connects the FGF21 mutant protein to the Fc region of the immunoglobulin.

13. The dual function protein of claim 12, wherein the linker is connected to the C-terminus of the Fc region of the immunoglobulin and the N-terminus of the FGF21 mutant protein.

14. The dual function protein of claim 12, wherein the linker is a peptide consisting of 10 to 30 amino acid residues.

15. The dual function protein of claim 14, wherein the linker comprises an amino acid sequence selected from the group consisting of SEQ ID NOs: 2 to 5.

16. The dual function protein of claim 1, wherein the Fc region of the immunoglobulin is any one of the Fc region of IgG1, IgG2, IgG3, IgG4 and IgD, or a hybrid Fc containing a combination thereof.

17. The dual function protein of claim 14, wherein the hybrid Fc comprises an IgG4 region and an IgD region.

18. The dual function protein of claim 1, wherein the dual function protein comprises the biologically active protein, the Fc region of the immunoglobulin and the FGF21 mutant protein, connected in this order from the N-terminus to the C-terminus.

19. The dual function protein of claim 18, wherein a linker is additionally connected between the Fc region of the immunoglobulin and the FGF21 mutant protein.

20. The dual function protein of claim 19, wherein the linker is connected to the C-terminus of the Fc region of the immunoglobulin and the N-terminus of the FGF21 mutant protein.

21. The dual function protein of claim 19, wherein the linker is a peptide consisting of 10 to 30 amino acid residues.

22. The dual function protein of claim 19, wherein the linker comprise an amino acid sequence selected from the group consisting of SEQ ID NOs: 2 to 5.

23. The dual function protein of claim 1, wherein the dual function protein comprise the amino acid sequence of SEQ ID NO: 65.

24. The dual function protein of claim 1, wherein the dual function protein comprises the amino acid sequence of SEQ ID NO: 66.

25. The dual function protein of claim 1, wherein the dual function protein has an amino acid sequence represented by SEQ ID NO: 67.

26. A pharmaceutical composition comprising the dual function protein according to claim 1 and a pharmaceutically acceptable carrier.

27. An isolated nucleic acid molecule encoding the fusion protein according to claim 1.

28. An expression vector comprising the nucleic acid molecule of claim 27.

29. A host cell comprising the nucleic acid molecule of claim 27.

30. A method for treating diabetes, obesity, dyslipidemia, metabolic syndrome, non-alcoholic fatty liver disease, non-alcoholic steatohepatitis or cardiovascular diseases in a subject in need thereof, comprising administering the pharmaceutical composition of claim 26 to the subject.

31. A method selected from the group consisting of:
reducing blood glucose level in a subject;
reducing body weight in a subject;
reducing triglyceride or low-density lipoprotein levels in a subject; and
improving insulin sensitivity in a subject
wherein the method comprising administering the pharmaceutical composition of claim 26 to the subject.

32. The method of claim 31, wherein the subject is obese.

33. The method of claim 32, wherein the subject has diabetes.

34. A method for inhibiting cAMP production in a subject in need thereof, comprising administering the composition of claim 26 to the subject.


SEQUENCE LISTING (83 sequences)

SEQ ID NO: 1 - protein, 181 aa, Homo sapiens
  REGION 1..181: note = human FGF21
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 2 - protein, 30 aa, synthetic construct
  REGION 1..30: note = linker
AKATTAPATTRNTGRGGEEKKKEKEKEEQE

SEQ ID NO: 3 - protein, 17 aa, synthetic construct
  REGION 1..17: note = linker
AKATTAPATTRNTGRGG

SEQ ID NO: 4 - protein, 15 aa, synthetic construct
  REGION 1..15: note = linker
GGGGSGGGGSGGGGS

SEQ ID NO: 5 - protein, 16 aa, synthetic construct
  REGION 1..16: note = linker
GGGGSGGGGSGGGGSA

SEQ ID NO: 6 - protein, 181 aa, synthetic construct
  REGION 1..181: note = FGF21 variant
HPIPDSSPLLQFGGQVRQRYLYTDDAQQTEAHLEIREDGTVGGAADQSPESLLQLKALKPGVIQILGVKTSRFLCQRPDGALYGSLHFDPEACSFREEIRPDGYNVYQSEAHGLPLHLPGNKSPHRDPAPRGPARFLPLPGLPPALPEPPGILAPQPPDVGSSDPLSMVGPSQGRSPSYAS

SEQ ID NO: 7 - protein, 182 aa, synthetic construct
  REGION 1..182: note = FGF21 variant
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVTGLEAVRSPSYAS

SEQ ID NO: 8 - protein, 182 aa, synthetic construct
  REGION 1..182: note = FGF21 variant
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVTGLEANRSPSYAS

SEQ ID NO: 9 - protein, 181 aa, synthetic construct
  REGION 1..181: note = FGF21 variant
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVNPSQGRSPSYAS

SEQ ID NO: 10 - protein, 181 aa, synthetic construct
  REGION 1..181: note = FGF21 variant
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQNRSPSYAS

SEQ ID NO: 11 - protein, 182 aa, synthetic construct
  REGION 1..182: note = FGF21 variant
HPIPDSSPLLQFGGQVRQRYLYTDDAQQTEAHLEIREDGTVGGAADQSPESLLQLKALKPGVIQILGVKTSRFLCQRPDGALYGSLHFDPEACSFREEIRPDGYNVYQSEAHGLPLHLPGNKSPHRDPAPRGPARFLPLPGLPPALPEPPGILAPQPPDVGSSDPLSMVTGLEAVRSPSYAS

SEQ ID NO: 12 - protein, 182 aa, synthetic construct
  REGION 1..182: note = FGF21 variant
HPIPDSSPLLQFGGQVRQRYLYTDDAQQTEAHLEIREDGTVGGAADQSPESLLQLKALKPGVIQILGVKTSRFLCQRPDGALYGSLHFDPEACSFREEIRPDGYNVYQSEAHGLPLHLPGNKSPHRDPAPRGPARFLPLPGLPPALPEPPGILAPQPPDVGSSDPLSMVTGLEANRSPSYAS

SEQ ID NO: 13 - protein, 181 aa, synthetic construct
  REGION 1..181: note = FGF21 variant
HPIPDSSPLLQFGGQVRQRYLYTDDAQQTEAHLEIREDGTVGGAADQSPESLLQLKALKPGVIQILGVKTSRFLCQRPDGALYGSLHFDPEACSFREEIRPDGYNVYQSEAHGLPLHLPGNKSPHRDPAPRGPARFLPLPGLPPALPEPPGILAPQPPDVGSSDPLSMVNPSQGRSPSYAS

SEQ ID NO: 14 - protein, 181 aa, synthetic construct
  REGION 1..181: note = FGF21 variant
HPIPDSSPLLQFGGQVRQRYLYTDDAQQTEAHLEIREDGTVGGAADQSPESLLQLKALKPGVIQILGVKTSRFLCQRPDGALYGSLHFDPEACSFREEIRPDGYNVYQSEAHGLPLHLPGNKSPHRDPAPRGPARFLPLPGLPPALPEPPGILAPQPPDVGSSDPLSMVGPSQNRSPSYAS

SEQ ID NO: 15 - protein, 181 aa, synthetic construct
  REGION 1..181: note = FGF21 variant
HPIPDSSPLLQFGGQVRQRYLYTDDAQQTEAHLEIREDGTVGGAADQSPESLLQLKALKPGVIQILGVKTSRFLCQRPDGALYGSLHFDPEACSFREEIRPDGYNVYQSEAHGLPLHLPGNKSPHRDPAPRGPARFLPLPGLPPALPEPPGILAPQPPDVGSSDPLSMVGPSQGRSPSYES

SEQ ID NO: 16 - protein, 182 aa, synthetic construct
  REGION 1..182: note = FGF21 variant
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVTGLEAVRSPSYES

SEQ ID NO: 17 - protein, 182 aa, synthetic construct
  REGION 1..182: note = FGF21 variant
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVTGLEANRSPSYES

SEQ ID NO: 18 - protein, 181 aa, synthetic construct
  REGION 1..181: note = FGF21 variant
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVNPSQGRSPSYES

SEQ ID NO: 19 - protein, 181 aa, synthetic construct
  REGION 1..181: note = FGF21 variant
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQNRSPSYES

SEQ ID NO: 20 - protein, 182 aa, synthetic construct
  REGION 1..182: note = FGF21 variant
HPIPDSSPLLQFGGQVRQRYLYTDDAQQTEAHLEIREDGTVGGAADQSPESLLQLKALKPGVIQILGVKTSRFLCQRPDGALYGSLHFDPEACSFREEIRPDGYNVYQSEAHGLPLHLPGNKSPHRDPAPRGPARFLPLPGLPPALPEPPGILAPQPPDVGSSDPLSMVTGLEAVRSPSYES

SEQ ID NO: 21 - protein, 182 aa, synthetic construct
  REGION 1..182: note = FGF21 variant
HPIPDSSPLLQFGGQVRQRYLYTDDAQQTEAHLEIREDGTVGGAADQSPESLLQLKALKPGVIQILGVKTSRFLCQRPDGALYGSLHFDPEACSFREEIRPDGYNVYQSEAHGLPLHLPGNKSPHRDPAPRGPARFLPLPGLPPALPEPPGILAPQPPDVGSSDPLSMVTGLEANRSPSYES

SEQ ID NO: 22 - protein, 181 aa, synthetic construct
  REGION 1..181: note = FGF21 variant
HPIPDSSPLLQFGGQVRQRYLYTDDAQQTEAHLEIREDGTVGGAADQSPESLLQLKALKPGVIQILGVKTSRFLCQRPDGALYGSLHFDPEACSFREEIRPDGYNVYQSEAHGLPLHLPGNKSPHRDPAPRGPARFLPLPGLPPALPEPPGILAPQPPDVGSSDPLSMVNPSQGRSPSYES

SEQ ID NO: 23 - protein, 181 aa, synthetic construct
  REGION 1..181: note = FGF21 variant
HPIPDSSPLLQFGGQVRQRYLYTDDAQQTEAHLEIREDGTVGGAADQSPESLLQLKALKPGVIQILGVKTSRFLCQRPDGALYGSLHFDPEACSFREEIRPDGYNVYQSEAHGLPLHLPGNKSPHRDPAPRGPARFLPLPGLPPALPEPPGILAPQPPDVGSSDPLSMVGPSQNRSPSYES

SEQ ID NO: 24 - protein, 229 aa, synthetic construct
  REGION 1..229: note = Human IgG4 Fc
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 25 - protein, 228 aa, synthetic construct
  REGION 1..228: note = Human IgG4 Fc variant
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 26 - protein, 223 aa, synthetic construct
  REGION 1..223: note = Hybrid Fc variant
ETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - protein, 435 aa, synthetic construct
  REGION 1..435: note = modified FGF21 variant connected to hybrid Fc
ETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAKATTAPATTRNTGRGGEEKKKEKEKEEQEHPIPDSSPLLQFGGQVRQRYLYTDDAQQTEAHLEIREDGTVGGAADQSPESLLQLKALKPGVIQILGVKTSRFLCQRPDGALYGSLHFDPEACSFREEIRPDGYNVYQSEAHGLPLHLPGNKSPHRDPAPRGPARFLPLPGLPPALPEPPGILAPQPPDVGSSDPLSMVTGLEAVRSPSYAS

SEQ ID NO: 28 - protein, 422 aa, synthetic construct
  REGION 1..422: note = modified FGF21 variant connected to hybrid Fc
ETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAKATTAPATTRNTGRGGHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVTGLEAVRSPSYAS

SEQ ID NO: 29 - protein, 420 aa, synthetic construct
  REGION 1..420: note = modified FGF21 variant connected to hybrid Fc
ETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVTGLEAVRSPSYAS

SEQ ID NO: 30 - protein, 420 aa, synthetic construct
  REGION 1..420: note = modified FGF21 variant connected to hybrid Fc
ETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVTGLEANRSPSYAS

SEQ ID NO: 31 - protein, 419 aa, synthetic construct
  REGION 1..419: note = modified FGF21 variant connected to hybrid Fc
ETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVNPSQGRSPSYAS

SEQ ID NO: 32 - protein, 419 aa, synthetic construct; identical to SEQ ID NO: 31
  REGION 1..419: note = modified FGF21 variant connected to hybrid Fc

SEQ ID NO: 33 - protein, 419 aa, synthetic construct
  REGION 1..419: note = modified FGF21 variant connected to hybrid Fc
ETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQNRSPSYAS

SEQ ID NO: 34 - protein, 419 aa, synthetic construct
  REGION 1..419: note = modified FGF21 variant connected to hybrid Fc
ETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 35 - protein, 420 aa, synthetic construct
  REGION 1..420: note = modified FGF21 variant connected to hybrid Fc
ETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFREEIRPDGYNVYQSEAHGLPLHLPGNKSPHRDPAPRGPARFLPLPGLPPALPEPPGILAPQPPDVGSSDPLSMVTGLEAVRSPSYAS

SEQ ID NO: 36 - protein, 420 aa, synthetic construct
  REGION 1..420: note = modified FGF21 variant connected to hybrid Fc
ETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFREEIRPDGYNVYQSEAHGLPLHLPGNKSPHRDPAPRGPARFLPLPGLPPALPEPPGILAPQPPDVGSSDPLSMVTGLEAVRSPSYES

SEQ ID NO: 37 - protein, 420 aa, synthetic construct
  REGION 1..420: note = modified FGF21 variant connected to hybrid Fc
ETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFREEIRPDGYNVYQSEAHGLPLHLPGNKSPHRDPAPRGPARFLPLPGLPPALPEPPGILAPQPPDVGSSDPLSMVTGLEANRSPSYES

SEQ ID NO: 38 - protein, 419 aa, synthetic construct
  REGION 1..419: note = modified FGF21 variant connected to hybrid Fc
ETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFREEIRPDGYNVYQSEAHGLPLHLPGNKSPHRDPAPRGPARFLPLPGLPPALPEPPGILAPQPPDVGSSDPLSMVNPSQGRSPSYAS

SEQ ID NO: 39 - protein, 419 aa, synthetic construct
  REGION 1..419: note = modified FGF21 variant connected to hybrid Fc
ETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFREEIRPDGYNVYQSEAHGLPLHLPGNKSPHRDPAPRGPARFLPLPGLPPALPEPPGILAPQPPDVGSSDPLSMVNPSQGRSPSYES

SEQ ID NO: 40 - protein, 423 aa, synthetic construct
  REGION 1..423: note = REG(Amgen)
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFRERLLEDGYNVYQSEAHGLPLHLPGNKSPHRDPAPRGPARFLPLPGLPPALPEPPGILAPQPPDVGSSDPLSMVGGSQGRSPSYES

SEQ ID NO: 41 - protein, 424 aa, synthetic construct
  REGION 1..424: note = FGF21 connected to Fc(lilly)
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSAHPIPDSSPLLQFGGQVRQRYLYTDDAQQTECHLEIREDGTVGCAADQSPESLLQLKALKPGVIQILGVKTSRFLCQRPDGALYGSLHFDPEACSFREDLKEDGYNVYQSEAHGLPLHLPGDKSPHRKPAPRGPARFLPLPGLPPALPEPPGILAPQPPDVGSSDPLRLVEPSQLRSPSFE

SEQ ID NO: 42 - protein, 31 aa, synthetic construct
  REGION 1..31: note = GLP-1
HAEGTFTSDVSSYLEGQAAKEFIAWLVKGRG

SEQ ID NO: 43 - protein, 31 aa, synthetic construct
  REGION 1..31: note = GLP-1 variant
HGEGTFTSDVSSYLEGQAAKEFIAWLVKGRG

SEQ ID NO: 44 - protein, 31 aa, synthetic construct
  REGION 1..31: note = GLP-1 variant
HGEGTFTSDVSSYLEEQAAKEFIAWLVKGRG

SEQ ID NO: 45 - protein, 31 aa, synthetic construct
  REGION 1..31: note = GLP-1 variant
HGEGTFTSDVSSYLEGQAAKEFIAWLVKGGG

SEQ ID NO: 46 - protein, 31 aa, synthetic construct
  REGION 1..31: note = GLP-1 variant
HGEGTFTSDVSSYLEEQAAKEFIAWLVKGGG

SEQ ID NO: 47 - protein, 245 aa, synthetic construct
  REGION 1..245: note = hybrid Fc5
RNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 48 - protein, 233 aa, synthetic construct
  REGION 1..233: note = hybrid Fc40
EKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 49 - protein, 276 aa, synthetic construct
  REGION 1..276: note = GLP-1 variant connected to hybrid Fc5
HGEGTFTSDVSSYLEGQAAKEFIAWLVKGRGRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 50 - protein, 264 aa, synthetic construct
  REGION 1..264: note = GLP-1 variant connected to hybrid Fc40
HGEGTFTSDVSSYLEGQAAKEFIAWLVKGRGEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 51 - protein, 276 aa, synthetic construct
  REGION 1..276: note = GLP-1 variant connected to hybrid Fc5
HGEGTFTSDVSSYLEEQAAKEFIAWLVKGRGRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 52 - protein, 264 aa, synthetic construct
  REGION 1..264: note = GLP-1 variant connected to hybrid Fc40
HGEGTFTSDVSSYLEEQAAKEFIAWLVKGRGEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 53 - protein, 276 aa, synthetic construct
  REGION 1..276: note = GLP-1 variant connected to hybrid Fc5
HGEGTFTSDVSSYLEGQAAKEFIAWLVKGGGRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 54 - protein, 264 aa, synthetic construct
  REGION 1..264: note = GLP-1 variant connected to hybrid Fc40
HGEGTFTSDVSSYLEGQAAKEFIAWLVKGGGEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 55 - protein, 276 aa, synthetic construct
  REGION 1..276: note = GLP-1 variant connected to hybrid Fc5
HGEGTFTSDVSSYLEEQAAKEFIAWLVKGGGRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 56 - protein, 264 aa, synthetic construct
  REGION 1..264: note = GLP-1 variant connected to hybrid Fc40
HGEGTFTSDVSSYLEEQAAKEFIAWLVKGGGEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 57 - protein, 275 aa, synthetic construct
  REGION 1..275: note = Dulaglutide
HGEGTFTSDVSSYLEEQAAKEFIAWLVKGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 58 - protein, 461 aa, synthetic construct
  REGION 1..461: note = GLP1(A2G)-HyFc40-GS3-FGF21(EIRP, TGLEAV)
HGEGTFTSDVSSYLEGQAAKEFIAWLVKGRGEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFREEIRPDGYNVYQSEAHGLPLHLPGNKSPHRDPAPRGPARFLPLPGLPPALPEPPGILAPQPPDVGSSDPLSMVTGLEAVRSPSYAS

SEQ ID NO: 59 - protein, 473 aa, synthetic construct
  REGION 1..473: note = GLP1(GE)-HyFc5-GS3-FGF21(EIRP, TGLEAV)
HGEGTFTSDVSSYLEEQAAKEFIAWLVKGRGRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFREEIRPDGYNVYQSEAHGLPLHLPGNKSPHRDPAPRGPARFLPLPGLPPALPEPPGILAPQPPDVGSSDPLSMVTGLEAVRSPSYAS

SEQ ID NO: 60 - protein, 461 aa, synthetic construct
  REGION 1..461: note = GLP1(GE)-HyFc40-GS3-FGF21(EIRP, TGLEAV)
HGEGTFTSDVSSYLEEQAAKEFIAWLVKGRGEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFREEIRPDGYNVYQSEAHGLPLHLPGNKSPHRDPAPRGPARFLPLPGLPPALPEPPGILAPQPPDVGSSDPLSMVTGLEAVRSPSYAS

SEQ ID NO: 61 - protein, 473 aa, synthetic construct
  REGION 1..473: note = GLP1(GG)-HyFc5-GS3-FGF21(EIRP, TGLEAV)
HGEGTFTSDVSSYLEGQAAKEFIAWLVKGGGRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFREEIRPDGYNVYQSEAHGLPLHLPGNKSPHRDPAPRGPARFLPLPGLPPALPEPPGILAPQPPDVGSSDPLSMVTGLEAVRSPSYAS

SEQ ID NO: 62 - protein, 461 aa, synthetic construct
  REGION 1..461: note = GLP1(GG)-HyFc40-GS3-FGF21(EIRP, TGLEAV)
HGEGTFTSDVSSYLEGQAAKEFIAWLVKGGGEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFREEIRPDGYNVYQSEAHGLPLHLPGNKSPHRDPAPRGPARFLPLPGLPPALPEPPGILAPQPPDVGSSDPLSMVTGLEAVRSPSYAS

SEQ ID NO: 63 - protein, 473 aa, synthetic construct
  REGION 1..473: note = GLP1(GEG)-HyFc5-GS3-FGF21(EIRP, TGLEAV)
HGEGTFTSDVSSYLEEQAAKEFIAWLVKGGGRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFREEIRPDGYNVYQSEAHGLPLHLPGNKSPHRDPAPRGPARFLPLPGLPPALPEPPGILAPQPPDVGSSDPLSMVTGLEAVRSPSYAS

SEQ ID NO: 64 - protein, 461 aa, synthetic construct
  REGION 1..461: note = GLP1(GEG)-HyFc40-GS3-FGF21(EIRP, TGLEAV)
HGEGTFTSDVSSYLEEQAAKEFIAWLVKGGGEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFREEIRPDGYNVYQSEAHGLPLHLPGNKSPHRDPAPRGPARFLPLPGLPPALPEPPGILAPQPPDVGSSDPLSMVTGLEAVRSPSYAS

SEQ ID NO: 65 - protein, 461 aa, synthetic construct
  REGION 1..461: note = GLP1(GEG)-HyFc40-GS3-FGF21(EIRP, TGLEAV, A180E)
HGEGTFTSDVSSYLEEQAAKEFIAWLVKGGGEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFREEIRPDGYNVYQSEAHGLPLHLPGNKSPHRDPAPRGPARFLPLPGLPPALPEPPGILAPQPPDVGSSDPLSMVTGLEAVRSPSYES

SEQ ID NO: 66 - protein, 461 aa, synthetic construct
  REGION 1..461: note = GLP1(GEG)-HyFc40-GS3-FGF21(EIRP, TGLEAN, A180E)
HGEGTFTSDVSSYLEEQAAKEFIAWLVKGGGEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFREEIRPDGYNVYQSEAHGLPLHLPGNKSPHRDPAPRGPARFLPLPGLPPALPEPPGILAPQPPDVGSSDPLSMVTGLEANRSPSYES

SEQ ID NO: 67 - protein, 460 aa, synthetic construct
  REGION 1..460: note = GLP1(GEG)-HyFc40-GS3-FGF21(EIRP, G170N, A180E)
HGEGTFTSDVSSYLEEQAAKEFIAWLVKGGGEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFREEIRPDGYNVYQSEAHGLPLHLPGNKSPHRDPAPRGPARFLPLPGLPPALPEPPGILAPQPPDVGSSDPLSMVNPSQGRSPSYES

SEQ ID NO: 68 - protein, 4 aa, synthetic construct
  REGION 1..4: note = FGF21 variant
EIRP

SEQ ID NO: 69 - protein, 6 aa, synthetic construct
  REGION 1..6: note = FGF21 variant
TGLEAV

SEQ ID NO: 70 - protein, 6 aa, synthetic construct
  REGION 1..6: note = FGF21 variant
TGLEAN

SEQ ID NO: 71 - DNA, 1383 bp, synthetic construct
  misc_feature 1..1383: note = nucleic acid molecule coding for DFD23
  source 1..1383: mol_type = other DNA
cacggcgaggggaccttcacaagcgacgtgtcctcttatctggaaggacaggccgctaaggagtttatcgcatggctcgtcaaaggcagaggcgaaaaggagaaggaagagcaggaggagagagaaaccaaaacacccgagtgtcccagtcacactcagcctctgggagtgtttctcttcccacctaagcccaaggatacccttatgatttctaggacacctgaggtgacctgcgtcgttgtggacgtgagtcaagaggacccagaggtccagtttaactggtatgttgacggcgtggaagtgcataatgcaaaaactaaaccccgcgaggaacaattcaattcaacctaccgggtcgtttctgtgttgacagtgctgcatcaagattggctgaacgggaaggagtataagtgtaaagtcagtaataagggactcccctctagtatcgaaaaaactatttcaaaggccaaaggccagcctagagagccacaggtgtacacccttcctccatcccaagaggagatgacaaagaaccaggtgtctctgacttgtctcgtgaaggggttctaccctagtgacatcgctgtcgaatgggagtcaaacggacagccagagaataattataagacaactcctcccgttctggattctgacggcagcttctttctgtactctaggcttactgtggacaaaagtcgctggcaagaagggaacgtcttttcatgttctgttatgcacgaggccttgcacaatcattatacacagaagtctctgagtctctcactgggcaaaggcgggggaggcagcgggggaggcgggtccggaggcgggggatctcatcccatccctgactccagtcctctcctgcaattcgggggccaagtccggcagcggtacctctacacagatgatgctcagcagacagaagcccacctggagatcagggaggatgggaccgtggggggcgctgctgaccagagccccgaaagtctcctgcagctgaaagccttgaagcctggagttattcaaatcttgggagtcaagactagtaggttcctgtgccagcggccagatggggccctgtatggatctctccattttgaccctgaggcctgcagcttccgggaggagatcagacccgacggatacaatgtttaccagtccgaagcccacggcctccctctgcatctgcccgggaacaagtctcctcaccgggaccctgcccccagaggacctgctcgcttcctgccactcccaggcctgccccccgcattgcctgagccacccggaatcctggccccccagccccctgatgtgggatcctctgaccctctgagcatggtgacaggcctggaggccgtgagaagccccagctacgcttcc

SEQ ID NO: 72 - DNA, 1419 bp, synthetic construct
  misc_feature 1..1419: note = nucleic acid molecule coding for DFD24
  source 1..1419: mol_type = other DNA
cacggcgaggggaccttcacaagcgacgtgtcctcttacctggaagagcaggccgctaaggaatttatcgcatggctcgtcaaaggaagagggaggaacaccggacggggcggggaagagaagaagaaagaaaaggagaaggaagagcaggaggagagagaaaccaaaacacccgagtgtcccagtcacactcagcctctgggagtgtttctcttcccacctaagcccaaggatacccttatgatttctaggacacctgaggtgacctgcgtcgttgtggacgtgagtcaagaggacccagaggtccagtttaactggtatgttgacggcgtggaagtgcataatgcaaaaactaaaccccgcgaggaacaattcaattcaacctaccgggtcgtttctgtgttgacagtgctgcatcaagattggctgaacgggaaggagtataagtgtaaagtcagtaataagggactcccctctagtatcgaaaaaactatttcaaaggccaaaggccagcctagagagccacaggtgtacacccttcctccatcccaagaggagatgacaaagaaccaggtgtctctgacttgtctcgtgaaggggttctaccctagtgacatcgctgtcgaatgggagtcaaacggacagccagagaataattataagacaactcctcccgttctggattctgacggcagcttctttctgtactctaggcttactgtggacaaaagtcgctggcaagaagggaacgtcttttcatgttctgttatgcacgaggccttgcacaatcattatacacagaagtctctgagtctctcactgggcaaaggcgggggaggcagcgggggaggcgggtccggaggcgggggatctcatcccatccctgactccagtcctctcctgcaattcgggggccaagtccggcagcggtacctctacacagatgatgctcagcagacagaagcccacctggagatcagggaggatgggaccgtggggggcgctgctgaccagagccccgaaagtctcctgcagctgaaagccttgaagcctggagttattcaaatcttgggagtcaagactagtaggttcctgtgccagcggccagatggggccctgtatggatctctccattttgaccctgaggcctgcagcttccgggaggagatcagacccgacggatacaatgtttaccagtccgaagcccacggcctccctctgcatctgcccgggaacaagtctcctcaccgggaccctgcccccagaggacctgctcgcttcctgccactcccaggcctgccccccgcattgcctgagccacccggaatcctggccccccagccccctgatgtgggatcctctgaccctctgagcatggtgacaggcctggaggccgtgagaagccccagctacgcttcc

SEQ ID NO: 73 - DNA, 1383 bp, synthetic construct
  misc_feature 1..1383: note = nucleic acid molecule coding for DFD25
  source 1..1383: mol_type = other DNA
cacggcgaggggaccttcacaagcgacgtgtcctcttacctggaagagcaggccgctaaggaatttatcgcatggctcgtcaaaggaagaggggaaaaggagaaggaagagcaggaggagagagaaaccaaaacacccgagtgtcccagtcacactcagcctctgggagtgtttctcttcccacctaagcccaaggatacccttatgatttctaggacacctgaggtgacctgcgtcgttgtggacgtgagtcaagaggacccagaggtccagtttaactggtatgttgacggcgtggaagtgcataatgcaaaaactaaaccccgcgaggaacaattcaattcaacctaccgggtcgtttctgtgttgacagtgctgcatcaagattggctgaacgggaaggagtataagtgtaaagtcagtaataagggactcccctctagtatcgaaaaaactatttcaaaggccaaaggccagcctagagagccacaggtgtacacccttcctccatcccaagaggagatgacaaagaaccaggtgtctctgacttgtctcgtgaaggggttctaccctagtgacatcgctgtcgaatgggagtcaaacggacagccagagaataattataagacaactcctcccgttctggattctgacggcagcttctttctgtactctaggcttactgtggacaaaagtcgctggcaagaagggaacgtcttttcatgttctgttatgcacgaggccttgcacaatcattatacacagaagtctctgagtctctcactgggcaaaggcgggggaggcagcgggggaggcgggtccggaggcgggggatctcatcccatccctgactccagtcctctcctgcaattcgggggccaagtccggcagcggtacctctacacagatgatgctcagcagacagaagcccacctggagatcagggaggatgggaccgtggggggcgctgctgaccagagccccgaaagtctcctgcagctgaaagccttgaagcctggagttattcaaatcttgggagtcaagactagtaggttcctgtgccagcggccagatggggccctgtatggatctctccattttgaccctgaggcctgcagcttccgggaggagatcagacccgacggatacaatgtttaccagtccgaagcccacggcctccctctgcatctgcccgggaacaagtctcctcaccgggaccctgcccccagaggacctgctcgcttcctgccactcccaggcctgccccccgcattgcctgagccacccggaatcctggccccccagccccctgatgtgggatcctctgaccctctgagcatggtgacaggcctggaggccgtgagaagccccagctacgcttcc

SEQ ID NO: 74 - DNA, 1419 bp, synthetic construct
  misc_feature 1..1419: note = nucleic acid molecule coding for DFD26
  source 1..1419: mol_type = other DNA
cacggcgaggggaccttcacaagcgacgtgtcctcttatctggaaggacaggccgctaaggagtttatcgcatggctcgtcaaaggcggcggcaggaacaccggacggggcggggaagagaagaagaaagaaaaggagaaggaagagcaggaggagagagaaaccaaaacacccgagtgtcccagtcacactcagcctctgggagtgtttctcttcccacctaagcccaaggatacccttatgatttctaggacacctgaggtgacctgcgtcgttgtggacgtgagtcaagaggacccagaggtccagtttaactggtatgttgacggcgtggaagtgcataatgcaaaaactaaaccccgcgaggaacaattcaattcaacctaccgggtcgtttctgtgttgacagtgctgcatcaagattggctgaacgggaaggagtataagtgtaaagtcagtaataagggactcccctctagtatcgaaaaaactatttcaaaggccaaaggccagcctagagagccacaggtgtacacccttcctccatcccaagaggagatgacaaagaaccaggtgtctctgacttgtctcgtgaaggggttctaccctagtgacatcgctgtcgaatgggagtcaaacggacagccagagaataattataagacaactcctcccgttctggattctgacggcagcttctttctgtactctaggcttactgtggacaaaagtcgctggcaagaagggaacgtcttttcatgttctgttatgcacgaggccttgcacaatcattatacacagaagtctctgagtctctcactgggcaaaggcgggggaggcagcgggggaggcgggtccggaggcgggggatctcatcccatccctgactccagtcctctcctgcaattcgggggccaagtccggcagcggtacctctacacagatgatgctcagcagacagaagcccacctggagatcagggaggatgggaccgtggggggcgctgctgaccagagccccgaaagtctcctgcagctgaaagccttgaagcctggagttattcaaatcttgggagtcaagactagtaggttcctgtgccagcggccagatggggccctgtatggatctctccattttgaccctgaggcctgcagcttccgggaggagatcagacccgacggatacaatgtttaccagtccgaagcccacggcctccctctgcatctgcccgggaacaagtctcctcaccgggaccctgcccccagaggacctgctcgcttcctgccactcccaggcctgccccccgcattgcctgagccacccggaatcctggccccccagccccctgatgtgggatcctctgaccctctgagcatggtgacaggcctggaggccgtgagaagccccagctacgcttcc

SEQ ID NO: 75 - DNA, 1383 bp, synthetic construct
  misc_feature 1..1383: note = nucleic acid molecule coding for DFD27
  source 1..1383: mol_type = other DNA
cacggcgaggggaccttcacaagcgacgtgtcctcttatctggaaggacaggccgctaaggagtttatcgcatggctcgtcaaaggcggcggcgaaaaggagaaggaagagcaggaggagagagaaaccaaaacacccgagtgtcccagtcacactcagcctctgggagtgtttctcttcccacctaagcccaaggatacccttatgatttctaggacacctgaggtgacctgcgtcgttgtggacgtgagtcaagaggacccagaggtccagtttaactggtatgttgacggcgtggaagtgcataatgcaaaaactaaaccccgcgaggaacaattcaattcaacctaccgggtcgtttctgtgttgacagtgctgcatcaagattggctgaacgggaaggagtataagtgtaaagtcagtaataagggactcccctctagtatcgaaaaaactatttcaaaggccaaaggccagcctagagagccacaggtgtacacccttcctccatcccaagaggagatgacaaagaaccaggtgtctctgacttgtctcgtgaaggggttctaccctagtgacatcgctgtcgaatgggagtcaaacggacagccagagaataattataagacaactcctcccgttctggattctgacggcagcttctttctgtactctaggcttactgtggacaaaagtcgctggcaagaagggaacgtcttttcatgttctgttatgcacgaggccttgcacaatcattatacacagaagtctctgagtctctcactgggcaaaggcgggggaggcagcgggggaggcgggtccggaggcgggggatctcatcccatccctgactccagtcctctcctgcaattcgggggccaagtccggcagcggtacctctacacagatgatgctcagcagacagaagcccacctggagatcagggaggatgggaccgtggggggcgctgctgaccagagccccgaaagtctcctgcagctgaaagccttgaagcctggagttattcaaatcttgggagtcaagactagtaggttcctgtgccagcggccagatggggccctgtatggatctctccattttgaccctgaggcctgcagcttccgggaggagatcagacccgacggatacaatgtttaccagtccgaagcccacggcctccctctgcatctgcccgggaacaagtctcctcaccgggaccctgcccccagaggacctgctcgcttcctgccactcccaggcctgccccccgcattgcctgagccacccggaatcctggccccccagccccctgatgtgggatcctctgaccctctgagcatggtgacaggcctggaggccgtgagaagccccagctacgcttcc

SEQ ID NO: 76 - DNA, 1419 bp, synthetic construct
  misc_feature 1..1419: note = nucleic acid molecule coding for DFD28
  source 1..1419: mol_type = other DNA
cacggcgaggggaccttcacaagcgacgtgtcctcttacctggaagagcaggccgctaaggaatttatcgcatggctcgtcaaaggaggcgggaggaacaccggacggggcggggaagagaagaagaaagaaaaggagaaggaagagcaggaggagagagaaaccaaaacacccgagtgtcccagtcacactcagcctctgggagtgtttctcttcccacctaagcccaaggatacccttatgatttctaggacacctgaggtgacctgcgtcgttgtggacgtgagtcaagaggacccagaggtccagtttaactggtatgttgacggcgtggaagtgcataatgcaaaaactaaaccccgcgaggaacaattcaattcaacctaccgggtcgtttctgtgttgacagtgctgcatcaagattggctgaacgggaaggagtataagtgtaaagtcagtaataagggactcccctctagtatcgaaaaaactatttcaaaggccaaaggccagcctagagagccacaggtgtacacccttcctccatcccaagaggagatgacaaagaaccaggtgtctctgacttgtctcgtgaaggggttctaccctagtgacatcgctgtcgaatgggagtcaaacggacagccagagaataattataagacaactcctcccgttctggattctgacggcagcttctttctgtactctaggcttactgtggacaaaagtcgctggcaagaagggaacgtcttttcatgttctgttatgcacgaggccttgcacaatcattatacacagaagtctctgagtctctcactgggcaaaggcgggggaggcagcgggggaggcgggtccggaggcgggggatctcatcccatccctgactccagtcctctcctgcaattcgggggccaagtccggcagcggtacctctacacagatgatgctcagcagacagaagcccacctggagatcagggaggatgggaccgtggggggcgctgctgaccagagccccgaaagtctcctgcagctgaaagccttgaagcctggagttattcaaatcttgggagtcaagactagtaggttcctgtgccagcggccagatggggccctgtatggatctctccattttgaccctgaggcctgcagcttccgggaggagatcagacccgacggatacaatgtttaccagtccgaagcccacggcctccctctgcatctgcccgggaacaagtctcctcaccgggaccctgcccccagaggacctgctcgcttcctgccactcccaggcctgccccccgcattgcctgagccacccggaatcctggccccccagccccctgatgtgggatcctctgaccctctgagcatggtgacaggcctggaggccgtgagaagccccagctacgcttcc

SEQ ID NO: 77 - DNA, 1383 bp, synthetic construct
  misc_feature 1..1383: note = nucleic acid molecule coding for DFD29
  source 1..1383: mol_type = other DNA
cacggcgaggggaccttcacaagcgacgtgtcctcttacctggaagagcaggccgctaaggaatttatcgcatggctcgtcaaaggaggcggggaaaaggagaaggaagagcaggaggagagagaaaccaaaacacccgagtgtcccagtcacactcagcctctgggagtgtttctcttcccacctaagcccaaggatacccttatgatttctaggacacctgaggtgacctgcgtcgttgtggacgtgagtcaagaggacccagaggtccagtttaactggtatgttgacggcgtggaagtgcataatgcaaaaactaaaccccgcgaggaacaattcaattcaacctaccgggtcgtttctgtgttgacagtgctgcatcaagattggctgaacgggaaggagtataagtgtaaagtcagtaataagggactcccctctagtatcgaaaaaactatttcaaaggccaaaggccagcctagagagccacaggtgtacacccttcctccatcccaagaggagatgacaaagaaccaggtgtctctgacttgtctcgtgaaggggttctaccctagtgacatcgctgtcgaatgggagtcaaacggacagccagagaataattataagacaactcctcccgttctggattctgacggcagcttctttctgtactctaggcttactgtggacaaaagtcgctggcaagaagggaacgtcttttcatgttctgttatgcacgaggccttgcacaatcattatacacagaagtctctgagtctctcactgggcaaaggcgggggaggcagcgggggaggcgggtccggaggcgggggatctcatcccatccctgactccagtcctctcctgcaattcgggggccaagtccggcagcggtacctctacacagatgatgctcagcagacagaagcccacctggagatcagggaggatgggaccgtggggggcgctgctgaccagagccccgaaagtctcctgcagctgaaagccttgaagcctggagttattcaaatcttgggagtcaagactagtaggttcctgtgccagcggccagatggggccctgtatggatctctccattttgaccctgaggcctgcagcttccgggaggagatcagacccgacggatacaatgtttaccagtccgaagcccacggcctccctctgcatctgcccgggaacaagtctcctcaccgggaccctgcccccagaggacctgctcgcttcctgccactcccaggcctgccccccgcattgcctgagccacccggaatcctggccccccagccccctgatgtgggatcctctgaccctctgagcatggtgacaggcctggaggccgtgagaagccccagctacgcttcc

SEQ ID NO: 78 - DNA, 1383 bp, synthetic construct
  misc_feature 1..1383: note = nucleic acid molecule coding for DFD69
  source 1..1383: mol_type = other DNA
cacggcgaggggaccttcacaagcgacgtgtcctcttacctggaagagcaggccgctaaggaatttatcgcatggctcgtcaaaggaggcggggaaaaggagaaggaagagcaggaggagagagaaaccaaaacacccgagtgtcccagtcacactcagcctctgggagtgtttctcttcccacctaagcccaaggatacccttatgatttctaggacacctgaggtgacctgcgtcgttgtggacgtgagtcaagaggacccagaggtccagtttaactggtatgttgacggcgtggaagtgcataatgcaaaaactaaaccccgcgaggaacaattcaattcaacctaccgggtcgtttctgtgttgacagtgctgcatcaagattggctgaacgggaaggagtataagtgtaaagtcagtaataagggactcccctctagtatcgaaaaaactatttcaaaggccaaaggccagcctagagagccacaggtgtacacccttcctccatcccaagaggagatgacaaagaaccaggtgtctctgacttgtctcgtgaaggggttctaccctagtgacatcgctgtcgaatgggagtcaaacggacagccagagaataattataagacaactcctcccgttctggattctgacggcagcttctttctgtactctaggcttactgtggacaaaagtcgctggcaagaagggaacgtcttttcatgttctgttatgcacgaggccttgcacaatcattatacacagaagtctctgagtctctcactgggcaaaggcgggggaggcagcgggggaggcgggtccggaggcgggggatctcatcccatccctgactccagtcctctcctgcaattcgggggccaagtccggcagcggtacctctacacagatgatgctcagcagacagaagcccacctggagatcagggaggatgggaccgtggggggcgctgctgaccagagccccgaaagtctcctgcagctgaaagccttgaagcctggagttattcaaatcttgggagtcaagactagtaggttcctgtgccagcggccagatggggccctgtatggatctctccattttgaccctgaggcctgcagcttccgggaggagatcagacccgacggatacaatgtttaccagtccgaagcccacggcctccctctgcatctgcccgggaacaagtctcctcaccgggaccctgcccccagaggacctgctcgcttcctgccactcccaggcctgccccccgcattgcctgagccacccggaatcctggccccccagccccctgatgtgggatcctctgaccctctgagcatggtgacaggcctggaggccgtgagaagccccagctacgagtcc

SEQ ID NO: 79 - DNA, 1383 bp, synthetic construct
  misc_feature 1..1383: note = nucleic acid molecule coding for DFD112
  source 1..1383: mol_type = other DNA
cacggcgaggggaccttcacaagcgacgtgtcctcttacctggaagagcaggccgctaaggaatttatcgcatggctcgtcaaaggaggcggggaaaaggagaaggaagagcaggaggagagagaaaccaaaacacccgagtgtcccagtcacactcagcctctgggagtgtttctcttcccacctaagcccaaggatacccttatgatttctaggacacctgaggtgacctgcgtcgttgtggacgtgagtcaagaggacccagaggtccagtttaactggtatgttgacggcgtggaagtgcataatgcaaaaactaaaccccgcgaggaacaattcaattcaacctaccgggtcgtttctgtgttgacagtgctgcatcaagattggctgaacgggaaggagtataagtgtaaagtcagtaataagggactcccctctagtatcgaaaaaactatttcaaaggccaaaggccagcctagagagccacaggtgtacacccttcctccatcccaagaggagatgacaaagaaccaggtgtctctgacttgtctcgtgaaggggttctaccctagtgacatcgctgtcgaatgggagtcaaacggacagccagagaataattataagacaactcctcccgttctggattctgacggcagcttctttctgtactctaggcttactgtggacaaaagtcgctggcaagaagggaacgtcttttcatgttctgttatgcacgaggccttgcacaatcattatacacagaagtctctgagtctctcactgggcaaaggcgggggaggcagcgggggaggcgggtccggaggcgggggatctcatcccatccctgactccagtcctctcctgcaattcgggggccaagtccggcagcggtacctctacacagatgatgctcagcagacagaagcccacctggagatcagggaggatgggaccgtggggggcgctgctgaccagagccccgaaagtctcctgcagctgaaagccttgaagcctggagttattcaaatcttgggagtcaagactagtaggttcctgtgccagcggccagatggggccctgtatggatctctccattttgaccctgaggcctgcagcttccgggaggagatcagacccgacggatacaatgtttaccagtccgaagcccacggcctccctctgcatctgcccgggaacaagtctcctcaccgggaccctgcccccagaggacctgctcgcttcctgccactcccaggcctgccccccgcattgcctgagccacccggaatcctggccccccagccccctgatgtgggatcctctgaccctctgagcatggtgacaggcctggaggccaacagaagccccagctacgagtcc

SEQ ID NO: 80 - DNA, 1380 bp, synthetic construct
  misc_feature 1..1380: note = nucleic acid molecule coding for DFD114
  source 1..1380: mol_type = other DNA
cacggcgaggggaccttcacaagcgacgtgtcctcttacctggaagagcaggccgctaaggaatttatcgcatggctcgtcaaaggaggcggggaaaaggagaaggaagagcaggaggagagagaaaccaaaacacccgagtgtcccagtcacactcagcctctgggagtgtttctcttcccacctaagcccaaggatacccttatgatttctaggacacctgaggtgacctgcgtcgttgtggacgtgagtcaagaggacccagaggtccagtttaactggtatgttgacggcgtggaagtgcataatgcaaaaactaaaccccgcgaggaacaattcaattcaacctaccgggtcgtttctgtgttgacagtgctgcatcaagattggctgaacgggaaggagtataagtgtaaagtcagtaataagggactcccctctagtatcgaaaaaactatttcaaaggccaaaggccagcctagagagccacaggtgtacacccttcctccatcccaagaggagatgacaaagaaccaggtgtctctgacttgtctcgtgaaggggttctaccctagtgacatcgctgtcgaatgggagtcaaacggacagccagagaataattataagacaactcctcccgttctggattctgacggcagcttctttctgtactctaggcttactgtggacaaaagtcgctggcaagaagggaacgtcttttcatgttctgttatgcacgaggccttgcacaatcattatacacagaagtctctgagtctctcactgggcaaaggcgggggaggcagcgggggaggcgggtccggaggcgggggatctcatcccatccctgactccagtcctctcctgcaattcgggggccaagtccggcagcggtacctctacacagatgatgctcagcagacagaagcccacctggagatcagggaggatgggaccgtggggggcgctgctgaccagagccccgaaagtctcctgcagctgaaagccttgaagcctggagttattcaaatcttgggagtcaagactagtaggttcctgtgccagcggccagatggggccctgtatggatctctccattttgaccctgaggcctgcagcttccgggaggagatcagacccgacggatacaatgtttaccagtccgaagcccacggcctccctctgcatctgcccgggaacaagtctcctcaccgggaccctgcccccagaggacctgctcgcttcctgccactcccaggcctgccccccgcattgcctgagccacccggaatcctggccccccagccccctgatgtgggatcctctgaccctctgagcatggtgaacccttcccagggcagaagccccagctacgagtcc

SEQ ID NO: 81 - protein, 4 aa, synthetic construct
  REGION 1..4: note = Position 98-101 of FGF21
LLLE

SEQ ID NO: 82 - protein, 5 aa, synthetic construct
  REGION 1..5: note = Position 170-174 of FGF21
GPSQG

SEQ ID NO: 83 - protein, 25 aa, synthetic construct
  REGION 1..25: note = Leader sequence
MDAMLRGLCCVLLLCGAVFVSPSHA